SEQUENCE LISTING (52 sequences)

SEQ ID NO: 1 - protein, 16 aa, Artificial Sequence
  Chemically Synthesized
  MISC_FEATURE (1)..(1): Ala, Ser, or Thr
  MISC_FEATURE (2)..(2): Met, Leu, Ala, Ile, or Val
  MISC_FEATURE (3)..(3): Met, Leu, Ala, Ile, or Val
  MISC_FEATURE (4)..(4): Phe or Ala
  MISC_FEATURE (5)..(5): Asp, Glu or Ala
  MISC_FEATURE (6)..(6): Ser, Thr, Ala, Glu, Asp, Gln, Asn, Phe, Tyr, Trp, Met, Leu, Ile, Val, Lys, His, Arg, or Aib
  MISC_FEATURE (7)..(7): Leu, Ile or Ala
  MISC_FEATURE (8)..(8): Leu, Ile or Ala
  MISC_FEATURE (9)..(9): Lys, Ala, Ser, Thr, Glu, Asp, Gln, Asn, Phe, Tyr, Trp, Met, Leu, Ile, Val, His, Arg, Aib, a lysine derivative, a ornithine derivative, or a 2,4-diaminobutyric acid derivative
  MISC_FEATURE (10)..(10): Glu, Gln, Ala, Ser, Thr, Asp, Asn, Phe, Tyr, Trp, Met, Leu, Ile, Val, Lys, His, Arg, or Aib
  MISC_FEATURE (11)..(11): Ala, Gly, Leu, Ile, or Aib
  MISC_FEATURE (12)..(12): Gln, Ala, Asn, Ser, Thr, Glu, Asp, Asn, Phe, Tyr, Trp, Met, Leu, Ile, Val, Lys, His, Arg, or Aib
  MISC_FEATURE (13)..(13): Gln, Arg, Lys, Ala, Ser, Thr, Glu, Asp, Asn, Phe, Tyr, Trp, Met, Leu, Ile, Val, or His
  MISC_FEATURE (14)..(14): Ser, Asn, Ala, Thr, Glu, Asp, Gln, Phe, Tyr, Trp, Met, Leu, Ile, Val, Lys, His, or Arg
  MISC_FEATURE (15)..(15): Leu
  MISC_FEATURE (16)..(16): His, or Arg
XXXXXXXXXXXXXXXX

SEQ ID NO: 2 - protein, 13 aa, Artificial Sequence
  Chemically Synthesized
  MISC_FEATURE (1)..(1): Met, Leu, Ala, Ile, or Val
  MISC_FEATURE (2)..(2): Asp, Ala, Glu, Asn, Gln, Ser, or Thr
  MISC_FEATURE (3)..(3): Phe or Ala
  MISC_FEATURE (4)..(4): Asp, Glu or Ala
  MISC_FEATURE (5)..(5): Ser, Thr, Ala, Glu, Asp, Gln, Asn, Phe, Tyr, Trp, Met, Leu, Ile, Val, Lys, His, Arg, or Aib
  MISC_FEATURE (6)..(6): Leu, Ile or Ala
  MISC_FEATURE (7)..(7): Leu, Ile or Ala
  MISC_FEATURE (8)..(8): Lys, Ala, Ser, Thr, Glu, Asp, Gln, Asn, Phe, Tyr, Trp, Met, Leu, Ile, Val, His, Arg, Aib, a lysine derivative, a ornithine derivative, or a 2,4-diaminobutyric acid derivative
  MISC_FEATURE (9)..(9): Glu, Gln, Ala, Ser, Thr, Asp, Asn, Phe, Tyr, Trp, Met, Leu, Ile, Val, Lys, His, Arg, or Aib
  MISC_FEATURE (10)..(10): Ala, Gly, Leu, Ile, or Aib
  MISC_FEATURE (11)..(11): Gln, Ala, Asn, Ser, Thr, Glu, Asp, Asn, Phe, Tyr, Trp, Met, Leu, Ile, Val, Lys, His, Arg, or Aib
  MISC_FEATURE (12)..(12): Gln, Arg, Lys, Ala, Ser, Thr, Glu, Asp, Asn, Phe, Tyr, Trp, Met, Leu, Ile, Val, or His
  MISC_FEATURE (13)..(13): Ser, Asn, Ala, Thr, Glu, Asp, Gln, Phe, Tyr, Trp, Met, Leu, Ile, Val, Lys, His, or Arg
XXXXXXXXXXXXX

SEQ ID NO: 3 - protein, 19 aa, Artificial Sequence
  Chemically Synthesized
SGGAMDFDSLLKEAQQSLH

SEQ ID NO: 4 - protein, 16 aa, Artificial Sequence
  Chemically Synthesized
AMDFDSLLKEAQQSLH

SEQ ID NO: 5 - protein, 17 aa, Artificial Sequence
  Chemically Synthesized
SLDFDSLLKEAQRSLRR

SEQ ID NO: 6 - protein, 17 aa, Artificial Sequence
  Chemically Synthesized
SLDFDDLLKQAQKNLRR

SEQ ID NO: 7 - protein, 13 aa, Artificial Sequence
  Chemically Synthesized
MDFDSLLKEAQQS

SEQ ID NO: 8 - protein, 13 aa, Artificial Sequence
  Chemically Synthesized
MAFDSLLKEAQQS

SEQ ID NO: 9 - protein, 13 aa, Artificial Sequence
  Chemically Synthesized
MDADSLLKEAQQS

SEQ ID NO: 10 - protein, 13 aa, Artificial Sequence
  Chemically Synthesized
MDFASLLKEAQQS

SEQ ID NO: 11 - protein, 13 aa, Artificial Sequence
  Chemically Synthesized
MDFDALLKEAQQS

SEQ ID NO: 12 - protein, 13 aa, Artificial Sequence
  Chemically Synthesized
MDFDSALKEAQQS

SEQ ID NO: 13 - protein, 13 aa, Artificial Sequence
  Chemically Synthesized
MDFDSLAKEAQQS

SEQ ID NO: 14 - protein, 13 aa, Artificial Sequence
  Chemically Synthesized
MDFDSLLAEAQQS

SEQ ID NO: 15 - protein, 13 aa, Artificial Sequence
  Chemically Synthesized
MDFDSLLKAAQQS

SEQ ID NO: 16 - protein, 13 aa, Artificial Sequence
  Chemically Synthesized
MDFDSLLKEGQQS

SEQ ID NO: 17 - protein, 13 aa, Artificial Sequence
  Chemically Synthesized
MDFDSLLKEAAQS

SEQ ID NO: 18 - protein, 13 aa, Artificial Sequence
  Chemically Synthesized
MDFDSLLKEAQAS

SEQ ID NO: 19 - protein, 13 aa, Artificial Sequence
  Chemically Synthesized
MAFDSLLKNAAAS

SEQ ID NO: 20 - protein, 13 aa, Artificial Sequence
  Chemically Synthesized
MAFDSLLKNAAQS

SEQ ID NO: 21 - protein, 13 aa, Artificial Sequence
  Chemically Synthesized
MAFDSLLKAAQQS

SEQ ID NO: 22 - protein, 13 aa, Artificial Sequence
  Chemically Synthesized
  MISC_FEATURE (9)..(9): Aib
MAFDSLLKXAQQS

SEQ ID NO: 23 - protein, 13 aa, Artificial Sequence
  Chemically Synthesized
MAFDSMLKAAQQS

SEQ ID NO: 24 - protein, 13 aa, Artificial Sequence
  Chemically Synthesized
  MISC_FEATURE (8)..(8): CrtDab
MDFDSLLXEAQQS

SEQ ID NO: 25 - protein, 21 aa, Artificial Sequence
  Chemically Synthesized
MDFDSLLKEAQQSRRRRRRRR

SEQ ID NO: 26 - protein, 16 aa, Artificial Sequence
  Chemically Synthesized
  MISC_FEATURE (1)..(1): Biot-Ahx-
  MISC_FEATURE (16)..(16): -NH2
AMDFDSLLKEAQQSLH

SEQ ID NO: 27 - protein, 16 aa, Artificial Sequence
  Chemically Synthesized
  MISC_FEATURE (1)..(1): Ac-
  MISC_FEATURE (16)..(16): -NH2
AMDFDSLLKEAQQSLH

SEQ ID NO: 28 - protein, 17 aa, Artificial Sequence
  Chemically Synthesized
  MISC_FEATURE (1)..(1): Ac-
  MISC_FEATURE (17)..(17): -NH2
SLDFDSLLKEAQRSLRR

SEQ ID NO: 29 - protein, 17 aa, Artificial Sequence
  Chemically Synthesized
  MISC_FEATURE (1)..(1): Ac-
  MISC_FEATURE (17)..(17): -NH2
SLDFDDLLKQAQKNLRR

SEQ ID NO: 30 - protein, 13 aa, Artificial Sequence
  Chemically Synthesized
  MISC_FEATURE (1)..(1): Ac-
  MISC_FEATURE (13)..(13): -NH2
MDFDSLLKEAQQS

SEQ ID NO: 31 - protein, 13 aa, Artificial Sequence
  Chemically Synthesized
  MISC_FEATURE (1)..(1): Ac-
  MISC_FEATURE (13)..(13): -NH2
MAFDSLLKEAQQS

SEQ ID NO: 32 - protein, 13 aa, Artificial Sequence
  Chemically Synthesized
  MISC_FEATURE (1)..(1): Ac-
  MISC_FEATURE (13)..(13): -NH2
MDADSLLKEAQQS

SEQ ID NO: 33 - protein, 13 aa, Artificial Sequence
  Chemically Synthesized
  MISC_FEATURE (1)..(1): Ac-
  MISC_FEATURE (13)..(13): -NH2
MDFASLLKEAQQS

SEQ ID NO: 34 - protein, 13 aa, Artificial Sequence
  Chemically Synthesized
  MISC_FEATURE (1)..(1): Ac-
  MISC_FEATURE (13)..(13): -NH2
MDFDALLKEAQQS

SEQ ID NO: 35 - protein, 13 aa, Artificial Sequence
  Chemically Synthesized
  MISC_FEATURE (1)..(1): Ac-
  MISC_FEATURE (13)..(13): -NH2
MDFDSALKEAQQS

SEQ ID NO: 36 - protein, 13 aa, Artificial Sequence
  Chemically Synthesized
  MISC_FEATURE (1)..(1): Ac-
  MISC_FEATURE (13)..(13): -NH2
MDFDSLAKEAQQS

SEQ ID NO: 37 - protein, 13 aa, Artificial Sequence
  Chemically Synthesized
  MISC_FEATURE (1)..(1): Ac-
  MISC_FEATURE (13)..(13): -NH2
MDFDSLLAEAQQS

SEQ ID NO: 38 - protein, 13 aa, Artificial Sequence
  Chemically Synthesized
  MISC_FEATURE (1)..(1): -Ac
  MISC_FEATURE (1)..(1): Ac-
  MISC_FEATURE (13)..(13): -NH2
MDFDSLLKAAQQS

SEQ ID NO: 39 - protein, 13 aa, Artificial Sequence
  Chemically Synthesized
  MISC_FEATURE (1)..(1): Ac-
  MISC_FEATURE (13)..(13): -NH2
MDFDSLLKEGQQS

SEQ ID NO: 40 - protein, 13 aa, Artificial Sequence
  Chemically Synthesized
  MISC_FEATURE (1)..(1): Ac-
  MISC_FEATURE (13)..(13): -NH2
MDFDSLLKEAAQS

SEQ ID NO: 41 - protein, 13 aa, Artificial Sequence
  Chemically Synthesized
  MISC_FEATURE (1)..(1): Ac-
  MISC_FEATURE (13)..(13): -NH2
MDFDSLLKEAQAS

SEQ ID NO: 42 - protein, 13 aa, Artificial Sequence
  Chemically Synthesized
  MISC_FEATURE (1)..(1): Ac-
  MISC_FEATURE (13)..(13): -NH2
MAFDSLLKNAAAS

SEQ ID NO: 43 - protein, 13 aa, Artificial Sequence
  Chemically Synthesized
  MISC_FEATURE (1)..(1): Ac-
  MISC_FEATURE (13)..(13): -NH2
MAFDSLLKNAAQS

SEQ ID NO: 44 - protein, 13 aa, Artificial Sequence
  Chemically Synthesized
  MISC_FEATURE (1)..(1): Ac-
  MISC_FEATURE (13)..(13): -NH2
MAFDSLLKAAQQS

SEQ ID NO: 45 - protein, 13 aa, Artificial Sequence
  Chemically Synthesized
  MISC_FEATURE (1)..(1): Ac-
  MISC_FEATURE (9)..(9): X=Aib
  MISC_FEATURE (13)..(13): -NH2
MAFDSLLKXAQQS

SEQ ID NO: 46 - protein, 13 aa, Artificial Sequence
  Chemically Synthesized
  MISC_FEATURE (1)..(1): Ac-
  MISC_FEATURE (13)..(13): -NH2
MAFDSMLKAAQQS

SEQ ID NO: 47 - protein, 13 aa, Artificial Sequence
  Chemically Synthesized
  MISC_FEATURE (1)..(1): Ac-
  MISC_FEATURE (8)..(8): X=CrtDab
  MISC_FEATURE (13)..(13): -NH2
MDFDSLLXEAQQS

SEQ ID NO: 48 - protein, 21 aa, Artificial Sequence
  Chemically Synthesized
  MISC_FEATURE (1)..(1): Ac-
  MISC_FEATURE (21)..(21): -NH2
MDFDSLLKEAQQSRRRRRRRR

SEQ ID NO: 49 - protein, 13 aa, Artificial Sequence
  Chemically Synthesized
  MISC_FEATURE (1)..(9): linker moiety linking residues 1 and 9
  MISC_FEATURE (1)..(1): Ac-
  MISC_FEATURE (13)..(13): -NH2
CAFDSLLKCAQQS

SEQ ID NO: 50 - protein, 13 aa, Artificial Sequence
  Chemically Synthesized
  MISC_FEATURE (1)..(9): linker moiety linking residues 1 and 9
CAFDSLLKCAQQS

SEQ ID NO: 51 - protein, 21 aa, Artificial Sequence
  Chemically Synthesized
  MISC_FEATURE (1)..(1): FITC-Ahx-
  MISC_FEATURE (21)..(21): -NH2
MDFDSLLKEAQQSRRRRRRRR

SEQ ID NO: 52 - protein, 8 aa, Artificial Sequence
  Chemically Synthesized
  MISC_FEATURE (1)..(1): FITC-Ahx-
  MISC_FEATURE (8)..(8): -NH2
RRRRRRRR

(19) United States
(12) Patent Application Publication
(10) Pub. No.: US 2018/0362587 A1
(43) Pub. Date: Dec. 20, 2018

(54) Compositions and Methods for Inhibiting CBP80 Binding to PGC1 Family of Co-Activators

(71) Applicant: University of Rochester, Rochester, NY (US)

(72) Inventors: Michael Gleghorn, Rochester, NY (US); Lynne Maquat, Rochester, NY (US); Rudi Fasan, Rochester, NY (US)

(21) Appl. No.: 16/060,109

(22) PCT Filed: Dec. 8, 2016
(86) PCT No.: PCT/US2016/000126

§ 371 Date: Jun. 7, 2018

Related U.S. Application Data
(60) Provisional application No. 62/264,671, filed on Dec. 8, 2015.
(60) Provisional application No. 62/264,677, filed on Dec. 8, 2015.

(51) Int. Cl. C07K 7/08; A61K 38/10; A61K 45/06; A61P 35/00

(52) U.S. Cl. CPC C07K 7/08; A61P 35/00; A61K 45/06; A61K 38/10

(57) ABSTRACT

The invention provides compositions and methods for inhibiting protein-protein interactions with cap-binding protein 80 (CBP80). In one embodiment, the invention provides compositions comprising linear and macrocylic peptides. In one embodiment, the invention provides methods for treating cancer, heart disease, autoimmune disorders, obesity, diabetes, or chronic inflammation disorders associated with the PGC1 family of co-activators.

### CROSS-REFERENCE TO RELATED APPLICATIONS

The present application is entitled to priority to U.S. Provisional Application No. 62/264,677, filed Dec. 8, 2015, and U.S. Provisional Application No. 62/264,671, filed Dec. 8, 2015, each of which is incorporated by reference herein in its entirety.

### STATEMENT REGARDING FEDERALLY SPONSORED RESEARCH OR DEVELOPMENT

This invention was made with government support under Grant No. R01 GM059614 awarded by the NIH. The government has certain rights in the invention.

### BACKGROUND OF THE INVENTION

Current standard-of-care therapies for treatment of estrogen receptor (ER)-positive breast cancers involve the use of ER antagonists (e.g. Tamoxifen) or aromatase inhibitors, which block the endogenous production of estrogen. Although tamoxifen has been used to treat breast cancer for more than 30 years, it shows diminished efficacy in the presence of anti-depressants (which are often co-prescribed during cancer treatment), and it increases the occurrence of uterine cancer and is associated high mortality risk. Major side effects of aromatase inhibitors include heart problems and osteoporosis, the latter leading to increased bone fractures in post-menopausal patients.

There is a knowledge gap regarding CBP80 interactions with PGC1 family of co-activators. There is a need in the art for inhibitors of the interaction between CBP80 and PGC1 family of co-activators. The present invention fills this need.

### SUMMARY OF THE INVENTION

In one aspect, the present invention provides macrocyclic peptide. In one embodiment, the macrocyclic peptide is represented by any one represented by any one of Formula (I)-Formula (V)

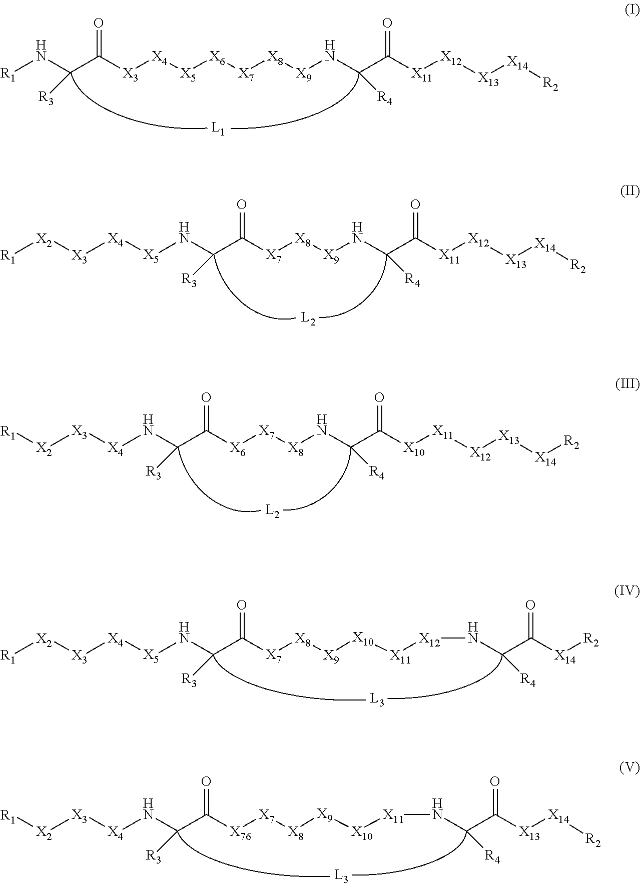

wherein

X₂ is Met, Leu, Ala, Ile, or Val;

X₃ is Asp, Ala, Glu, Asn, Gln, Ser, or Thr;

X₄ is Phe or Ala;

X₅ is Asp, Glu or Ala;

X₆ is Ser, Thr, Ala, Glu, Asp, Gln, Asn, Phe, Tyr, Trp, Met, Leu, Ile, Val, Lys, His, Arg, or Aib;

X₇ is Leu, Ile or Ala;

X₈ is Leu, Ile or Ala;

X₉ is Lys, Ala, Ser, Thr, Glu, Asp, Gln, Asn, Phe, Tyr, Trp, Met, Leu, Ile, Val, His, Arg, Aib, a lysine derivative, a omithine derivative, or a 2,4-diaminobutyric acid derivative;

X₁₀ is Glu, Gln, Ala, Ser, Thr, Asp, Asn, Phe, Tyr, Trp, Met, Leu, Ile, Val, Lys, His, Arg, or Aib;

X₁₁ is Ala, Gly, Leu, Ile, or Aib;

X₁₂ is Gln, Ala, Asn, Ser, Thr, Glu, Asp, Asn, Phe, Tyr, Trp, Met, Leu, Ile, Val, Lys, His, Arg, or Aib;

X₁₃ is Gln, Arg, Lys, Ala, Ser, Thr, Glu, Asp, Asn, Phe, Tyr, Trp, Met, Leu, Ile, Val, or His;

X₁₄ is Ser, Asn, Ala, Thr, Glu, Asp, Gln, Phe, Tyr, Trp, Met, Leu, Ile, Val, Lys, His, or Arg;

R₁ is hydrogen, an acetyl group, or a label molecule, wherein the label molecule is optionally comprises a spacer unit;

R₂ is a free carboxylic group or an amide group;

R₃ is hydrogen or methyl group;

R₄ is hydrogen or methyl group;

L₁ is a linker unit, such that the linear dimension between the Cα carbon atoms connected by the linker unit is between about 10 and 18 Angstrom units;

L₂ is —(CH₂)₃CH═CH(CH₂)₃— or —(CH₂)₈—; and

L₃ is —(CH₂)₆CH═CH(CH₂)₃— or —(CH₂)₁₁—.

In one embodiment, X₉ is selected from the group consisting of a lysine, ornithine and a 2,4-diaminobutyric acid derivative bearing a side-chain electrophilic group capable of reacting with cysteine.

In one embodiment, the electrophilic group capable of reacting with cysteine is selected from the group consisting

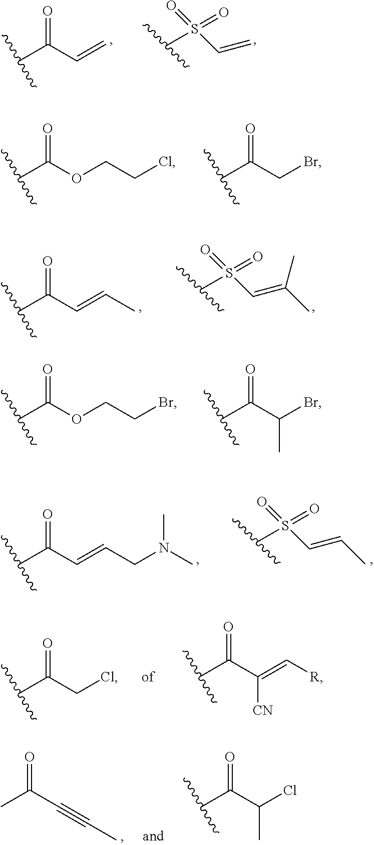

wherein R is selected from the group consisting of a C₃-C₂₀ alkyl, C₃-C₂₀ heteroalkyl, C₆-C₁₀ aryl, and C₆-C₁₀ heteroaryl group.

In one embodiment, L₁ is selected from the group consisting of:

—CH₂S(CH₂)ₙSCH₂—, wherein n is an integer number comprised between 4 and 8;

—CH₂S CH₂CH═CHCH₂SCH₂—;

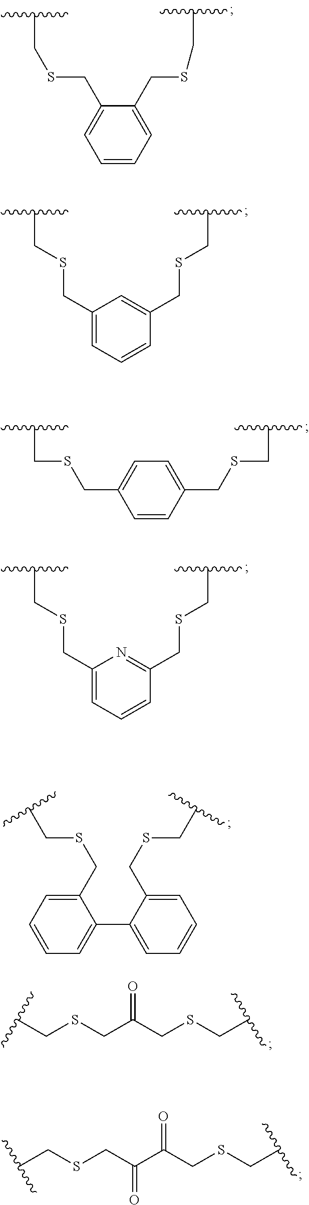

—(CH₂)ₘNHCO(CH₂)ₙ—, wherein m is an integer number between 2 and 4, and n is an integer number between 1 and 2;

—(CH₂)ₙCONH(CH₂)ₘ—, wherein m is an integer number between 2 and 4, and n is an integer number between 1 and 2;

—(CH₂)ₘCH═CH(CH₂)ₙ—, wherein m is an integer number between 1 and 6, and n is an integer number between 1 and 6;

—(CH₂)ₘCH₂CH₂(CH₂)ₙ—, wherein m is an integer number between 1 and 6, and n is an integer number between 1 and 6;

—(CH₂)ₘC≡C(CH₂)ₙ—, wherein m is an integer number between 1 and 6, and n is an integer number between 1 and 6; and

—(CH₂)ₘ(triazole)(CH₂)ₙ—, wherein m is an integer number between 1 and 6, and n is an integer number between 1 and 6.

In one embodiment, the cyclic peptide is selected from the group consisting of

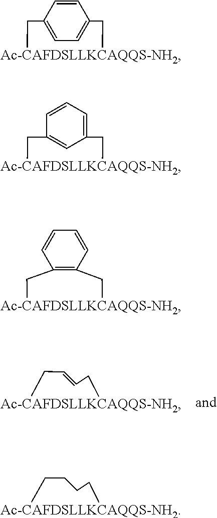

In one embodiment, the macrocyclic peptide comprises a group on the N-terminus of the peptide, wherein the group is selected from the group consisting of a hydrogen, an acetyl, and a label. In one embodiment, the macrocyclic peptide comprises a group on the C-terminus of the peptide, wherein the group is selected from the group consisting of a free carboxylic group, an amide group, or a cell penetrating peptide.

In one embodiment, the label is selected from the group consisting of an affinity label molecule, a photoaffinity label, a dye, a chromophore, a fluorescent molecule, a phosphorescent molecule, a chemiluminascent molecule, an energy transfer agent, a photocrosslinker molecule, a redox-active molecule, an isotopic label molecule, a spin label molecule, a metal chelator, a metal-comprising moiety, a heavy atom-comprising-moiety, a radioactive moiety, a contrast agent molecule, a MRI contrast agent, an isotopically labeled molecule, a PET agent, a polypeptide, a cell penetrating polypeptide, a carbohydrate, a polynucleotide, a peptide nucleic acid, a fatty acid, a lipid, biotin, a biotin analogue, a polymer, a small molecule, a drug or drug candidate, a cytotoxic molecule, a solid support, a surface, a resin, a nanoparticle, a quantum dot and any combination thereof.

In one embodiment, the group is a cell penetrating peptide and wherein the cell penetrating peptide is attached to the C-terminus of the peptide optionally via a spacer.

In one embodiment, the macrocyclic peptide binds human CBP80. In one embodiment, the macrocyclic peptide inhibits the binding of CBP80 to a binding partner. In one embodiment, the binding partner is a member of the PGC1 family of co-activators.

In another aspect, the present invention provides a peptide that inhibits the binding of CBP80 to a binding partner. In one embodiment, the peptide comprises a sequence selected from the group consisting of X₁X₂X₃X₄X₅X₆X₇X₈X₉X₁₀X₁₁X₁₂X₁₃X₁₄X₁₅X₁₆ (SEQ ID NO: 1) and X₂X₃X₄X₅X₆X₇X₈X₉X₁₀X₁₁X₁₂X₁₃X₁₄ (SEQ ID NO:2),

wherein X₁ is Ala, Ser, or Thr;

X₂ is Met, Leu, Ala, Ile, or Val;

X₃ is Asp, Ala, Glu, Asn, Gln, Ser, or Thr;

X₄ is Phe or Ala;

X₅ is Asp, Glu or Ala;

X₆ is Ser, Thr, Ala, Glu, Asp, Gln, Asn, Phe, Tyr, Trp, Met, Leu, Ile, Val, Lys, His, Arg, or Aib;

X₇ is Leu, Ile or Ala;

X₈ is Leu, Ile or Ala;

X₉ is Lys, Ala, Ser, Thr, Glu, Asp, Gln, Asn, Phe, Tyr, Trp, Met, Leu, Ile, Val, His, Arg, Aib, a lysine derivative, a omithine derivative, or a 2,4-diaminobutyric acid derivative;

X₁₀ is Glu, Gln, Ala, Ser, Thr, Asp, Asn, Phe, Tyr, Trp, Met, Leu, Ile, Val, Lys, His, Arg, or Aib;

X₁₁ is Ala, Gly, Leu, Ile, or Aib;

X₁₂ is Gln, Ala, Asn, Ser, Thr, Glu, Asp, Asn, Phe, Tyr, Trp, Met, Leu, Ile, Val, Lys, His, Arg, or Aib;

X₁₃ is Gln, Arg, Lys, Ala, Ser, Thr, Glu, Asp, Asn, Phe, Tyr, Trp, Met, Leu, Ile, Val, or His;

X₁₄ is Ser, Asn, Ala, Thr, Glu, Asp, Gln, Phe, Tyr, Trp, Met, Leu, Ile, Val, Lys, His, or Arg;

X₁₅ is Leu; and

X₁₆ is His, or Arg.

In one embodiment, X₉ is selected from the group consisting of a lysine, omithine and a 2,4-diaminobutyric acid derivative bearing a side-chain electrophilic group capable of reacting with cysteine.

In one embodiment, the electrophilic group capable of reacting with cysteine is selected from the group consisting

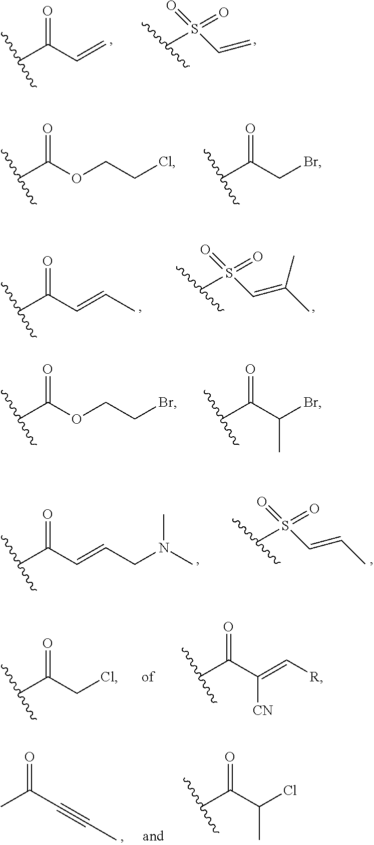

wherein R is selected from the group consisting of a C₃-C₂₀ alkyl, C₃-C₂₀ heteroalkyl, C₆-C₁₀ aryl, and C₆-C₁₀ heteroaryl group.

In one embodiment, the peptide comprises a group on the N-terminus of the peptide, wherein the group is selected from the group consisting of a hydrogen, an acetyl, and a label. In one embodiment, the peptide comprises a group on the C-terminus of the peptide, wherein the group is selected from the group consisting of a free carboxylic group, an amide group, or a cell penetrating peptide.

In one embodiment, label is selected from the group consisting of an affinity label molecule, a photoaffinity label, a dye, a chromophore, a fluorescent molecule, a phosphorescent molecule, a chemiluminascent molecule, an energy transfer agent, a photocrosslinker molecule, a redox-active molecule, an isotopic label molecule, a spin label molecule, a metal chelator, a metal-comprising moiety, a heavy atom-comprising-moiety, a radioactive moiety, a contrast agent molecule, a MRI contrast agent, an isotopically labeled molecule, a PET agent, a polypeptide, a cell penetrating polypeptide, a carbohydrate, a polynucleotide, a peptide nucleic acid, a fatty acid, a lipid, biotin, a biotin analogue, a polymer, a small molecule, a drug or drug candidate, a cytotoxic molecule, a solid support, a surface, a resin, a nanoparticle, a quantum dot and any combination thereof.

In one embodiment, the group is a cell penetrating peptide and wherein the cell penetrating peptide is attached to the C-terminus of the peptide optionally via a spacer.

In one embodiment, the peptide binds human CBP80. In one embodiment, the binding partner is a member of the PGC1 family of co-activators.

In one embodiment, the peptide comprises a sequence selected from the group consisting of AMDFDSLLKEAQQSLH (SEQ ID NO:3), AMDFDSLLKEAQQSLH (SEQ ID NO:4), SLDFDSLLKEAQRSLRR (SEQ ID NO:5), SLDFDDLLKQAQKNLRR (SEQ ID NO:6), MDFDSLLKEAQQS (SEQ ID NO:7), MAFDSLLKEAQQS (SEQ ID NO:8), MDADSLLKEAQQS (SEQ ID NO:9), MDFASLLKEAQQS (SEQ ID NO:10), MDFDALLKEAQQS (SEQ ID NO:11), MDFDSALKEAQQS (SEQ ID NO: 12), MDFDSLAKEAQQS (SEQ ID NO: 13), MDFDSLLAEAQQS (SEQ ID NO:14), MDFDSLLKAAQQS (SEQ ID NO:15), MDFDSLLKEGQQS (SEQ ID NO: 16), MDFDSLLKEAAQS (SEQ ID NO: 17), MDFDSLLKEAQAS (SEQ ID NO: 18), MAFDSLLKNAAAS (SEQ ID NO: 19), MAFDSLLKNAAQS (SEQ ID NO:20), MAFDSLLKAAQQS (SEQ ID NO:21), MAFDSLLK(Aib)AQQS (SEQ ID NO:22), MAFDSMLKAAQQS (SEQ ID NO:23), MDFDSLL(CrtDab)EAQQS (SEQ ID NO:24), and MDFDSLLKEAQQSRRRRRRRR (SEQ ID NO:25).

In another aspect, the invention provides a method for treating or preventing a disease or disorder in a subject in need thereof. In one embodiment, the method comprises administering to the subject an effective amount of a composition comprising a peptide or macrocyclic peptide of the invention.

In one embodiment, the disease or disorder is cancer, heart disease, autoimmune disorders, obesity, diabetes, or chronic inflammation disorders. In one embodiment, the cancer is breast cancer. In one embodiment, the breast cancer is ER negative breast cancer.

In one embodiment, the composition further comprises a pharmaceutically acceptable carrier.

In one embodiment, the method further comprises administering to the subject a second agent. In one embodiment, the second agent is an immunomodulatory agent, an antineoplastic agent, an anti-angiogenic agent or an anti-diabetic agent.

In invention also provides a method for inhibiting the interaction between CBP80 and a PGC1 family of co-activator in a subject in need thereof. In one embodiment, the method comprises administering to the subject an effective amount of a composition comprising a peptide or macrocyclic peptide of the invention.

In one embodiment, the PGC1 family of co-activator is selected from the group consisting of PRC, PGC1α and PGC1β.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1D, depicts experiential results demonstrating that the C-terminus of PGC1β interacts with CBP80 in vitro. FIG. 1A depicts a schematic of known or putative functional regions of PGC1β (upper) or E. coli-produced GST-PGC1β deletion variants and their binding to baculovirus-produced CBP80-TEV-HIS (lower). FIG. 1B and FIG. 1C depicts SDS-PAGE separation and Coomassie blue-staining of GST pull-downs. FIG. 1D depicts SDS-PAGE separation and Coomassie blue-staining identifying PGC1β variants that inhibit CBP80-TEV-HIS pull-down.

FIG. 2 depicts an X-ray crystal structure model (mid-refinement) of the PGC1β-CBP80-CBP20-cap complex at 2.7 Å resolution. Proteins and cap are indicated by color legend. Top left inset box zooms in on PGC1β (synthetic peptide) residues 1009-1023 and illustrates that identically conserved residues (bottom alignment; yellow) in PGC1 paralogs are residues that mediate the direct CBP80 interaction.

FIG. 3 depicts the X-ray crystal structure of the PGC1β-CBP80-CBP20-cap complex at 2.7 Å resolution. Proteins and RNA 5′ cap are denoted in the colored legend. The 3-dimensional box to the left of the legend zooms in on PGC1β amino acids 1010-1020 (dark blue), which constitute the α-helical region of PGC1β that are mimicked by the peptidomimetics (i.e. α-MOrPH inhibitors) described herein. In this box, amino acid side-chains of PGC1β (also dark blue) and CBP80 (green) are represented as “sticks”.

FIG. 4, comprising FIG. 4A and FIG. 4B depicts inhibitory α-MOrPHs. FIG. 4A depicts the chemical structures of first-generation α-MOrPH-based PGC1β peptidomimetics as inhibitors of the CBP80-PGC1β interaction. The structure of each non-peptidic linker that connects the side-chain of the peptide with its C-terminus is provided in the box. Each α-MOrPH inhibitor contains what are defined using X-ray crystallography as the CBP80-binding peptide of PGC1β (DFDSLLKEAQQ) and are acetylated (“Ac”) at the N-terminus. Fluorescein (FITC)-labeled analogs such as FITC-PGC1β-M4 will be used to assess cell permeability. FIG. 4B depicts a 3-dimensional model of a representative α-MOrPH structure.

FIG. 5, comprising FIG. 5A and FIG. 5B depicts the results of a dissociation assay of a FITC-labeled CBP80-PGC1β peptide complex. FIG. 5A depicts a schematic description of the fluorescence polarization-based assay for measuring the inhibitory potency (IC50) in vitro of an α-MOrPH. α-MOrPHs will mimic the PGC1β α-helix that binds CBP80. A constant amount of fluorescently labeled PGC1β peptide (i.e. FITC-Ahx-AMDFDSLLKEAQQSLH, where Ahx=6-amino-caproic acid) and a constant amount of CBP80 protein is mixed and incubated with increasing amounts of inhibitory α-MOrPH. This will result in an increase in fluorescence anisotropy when the complex of FITC-labeled PGC1β and CBP80 is dissociated. FIG. 5B depicts a representative dose-dependent inhibition curve as measured upon titration of Biot-PGC1β peptide (Biot-Ahx-SGG-AMDFDSLLKEAQQSLH-NH₂₎ in the presence of a fixed concentration of CBP80/CBP20 complex and fluorescein-conjugated PGC1β-derived peptide (FITC-Ahx-AMDFDSLLKEAQQSLH-NH₂).

FIG. 6, comprising FIG. 6A and FIG. 6B depicts experimental results demonstrating the C-terminus of PGC1β and PGC1α interacts with CBP80 in vitro. FIG. 6A depicts diagrams of known or putative functional regions of PGC1β relative to PGC1α. Lines below diagrams denote GST-tagged regions of each tested for CBP80 binding. FIG. 6B depicts SDS-PAGE separation and Coomassie blue-staining of GST pull-downs of the denoted region of PGC1β of PGC1α to identify binding to CBP80-TEV-HIS.

FIG. 7, comprising FIG. 7A and FIG. 7B depicts the refined crystal structure of PGC1β CBM-CBP80-CBP20-m⁷GpppG. FIG. 7A depicts the crystal structure of the PGC1β CBP80-binding motif (CBM) bound to the cap-binding complex (CBC). The CBM is defined as amino acids 1011-1019 (identical to PGC1α amino acids 785-793). FIG. 7B depicts a comparison between the structure in FIG. 7A and the structure of the CBC bound to importin-α. Since importin-α remains bound the CBP80 throughout its lifetime in both the nucleus and the cytoplasm, it is significant that the PGC1β binding site on CBP80 does not overlap the importin-α binding site on CBP80.

FIG. 8, comprising FIG. 8A through FIG. 8F depicts experimental results demonstrating that PGC1α co-immunoprecipitates with CBP80 and binds Idh3b, PJk1 and Sirt5 gene loci enhancer and protein-encoding RNA transcripts in C2Cl2 cells. FIG. 8A depicts western blotting of lysates of mouse C2Cl2 myoblasts (MBs), which were (+) or were not (−) crosslinked using formaldehyde prior to lysis, before (−) or after immunoprecipitation (IP) using anti (α)-PCG1α or, as a control, rabbit (r)IgG in the presence (+) or absence (−) of RNase I. The four leftmost lanes are serial dilutions of lysates prior to IP. FIG. 8B depicts western blotting of lysates of mouse C2Cl2 myoblasts (MBs), which were (+) or were not (−) crosslinked using formaldehyde prior to lysis, before (−) or after immunoprecipitation (IP) using anti (α)-CBP80 or, as a control, rabbit (r)IgG in the presence (+) or absence (−) of RNase I. FIG. 8C depicts western blotting of lysates of mouse C2Cl2 myoblasts (MBs), which were (+) or were not (−) crosslinked using formaldehyde prior to lysis, before (−) or after immunoprecipitation (IP) using anti (α)-CBP20 or, as a control, rabbit (r)IgG in the presence (+) or absence (−) of RNase I. FIG. 8D depicts histogram representation of RT-qPCR quantitations of eRNA from each of the three gene loci after (+) IP relative to before IP. FIG. 8E depicts histogram representation of RT-qPCR quantitations of pre-mRNA from each of the three gene loci after (+) IP relative to before IP. FIG. 8E depicts histogram representation of RT-qPCR quantitations of mRNA from each of the three gene loci after (+) IP relative to before IP.

FIG. 9, comprising FIG. 9A through FIG. 9H, depicts experimental results demonstrating co-activation by PGC1α depends on its CBM.

FIG. 9A depicts diagrams of human (h) PCG1α wild-type (WT) and deletion variants. FIG. 9B depicts western blotting of lysates of mouse (m) C2Cl2 cells, in which Pcg1α was knocked-down (KD) and replaced by the specified FLAG-tagged PCG1α variant, or FLAG alone (−), before (−) or after (+) IP using anti-FLAG in the presence or absence of RNase I. FIG. 9C depicts histogram representation of RT-qPCR quantitations of eRNA from each of the three gene loci normalized to the level of Hprt mRNA using lysates prior to IP. FIG. 9D depicts histogram representation of RT-qPCR quantitations of pre-mRNA from each of the three gene loci normalized to the level of Hprt mRNA using lysates prior to IP. FIG. 9E depicts histogram representation of RT-qPCR quantitations of mRNA from each of the three gene loci normalized to the level of Hprt mRNA using lysates prior to IP. FIG. 9F depicts histogram representation of RT-qPCR quantitations of eRNA from each of the three gene loci normalized to the level of Hprt mRNA using lysates after IP. FIG. 9G depicts histogram representation of RT-qPCR quantitations of pre-mRNA from each of the three gene loci normalized to the level of Hprt mRNA using lysates after IP. FIG. 9H depicts histogram representation of RT-qPCR quantitations of mRNA from each of the three gene loci normalized to the level of Hprt mRNA using lysates after IP.

FIG. 10, comprising FIG. 10A through FIG. 10E depicts experimental results demonstrating that PGC1α binds specifically to the 5′ end of RNAs. FIG. 10A depicts a schematic for DNA oligonucleotide-directed cleavage of cellular pre-mRNP and mRNP. Blue oligonucleotides show cleavage position directly downstream of the cap. FIG. 10B depicts western blotting of lysates of C2Cl2 cells, in which Pcg1α was knocked-down (KD) and replaced by the specified FLAG-tagged PCG1α variant, or FLAG alone (−), before or after IP using anti-FLAG beads in the presence or absence of RNase H. FIG. 10C depicts histogram representation of RT-qPCR showing the efficiency of cleavage. Cleavage was measured using a primer that overlapped the cleavage site so that no signal is generated from cleaved RNA. FIG. 10D depicts histogram representation of RT-qPCR quantitations of pre-mRNA from each of the three gene loci normalized using lysates prior to IP. FIG. 10E depicts histogram representation of RT-qPCR quantitations of mRNA from each of the three gene loci normalized using lysates prior to IP.

FIG. 11 depicts experimental results demonstrating that the CBM of PGC1α is required for efficient myogenic differentiation. Shown is western blotting of lysates of mouse (m) C2Cl2 cells, in which Pcg1α was knocked down (KD) and replaced by FLAG-PCG1α WT or FLAG-PCG1α ΔCBM, after 0, 1, 3, and 5 days in differentiation medium.

FIG. 12, comprising FIG. 12A and FIG. 12B, depict experimental results demonstrating cell permeability and localization of a representative fluorescein-conjugated PGC1β-derived peptide (FITC-Ahx-MDFDSLLKEAQQSRRRRRRRR-NH₂(SEQ ID NO:51)). FIG. 12A depicts micrographs of HeLa cells treated with 2 μM of the peptide after DAPI staining. Overlap shows both blue (DAPI) and green (FITC). FIG. 12B depicts the analysis of the populations of cells treated with increasing concentrations of fluorescein-conjugated PGC1β-derived peptide sorted by flow cytometry. Gray peak, control cell population; blue peak; 2 μM inhibitor; pink peak, 5 μm inhibitor; red peak, 10 μm inhibitor. Cells showed increasing fluorescence, indicating increasing inhibitor uptake.

FIG. 13 depicts a plot of fluorescence polarization signal at varying concentration of the CBP80 as measured upon titration of CBP80/CBP20 complex in the presence of the fluorescein-conjugated PGC1β-derived peptide (FITC-Ahx-AMDFDSLLKEAQQSLH-NH₂).

### DETAILED DESCRIPTION

In one aspect, the present invention provides compositions and methods for inhibiting protein-protein interactions with cap-binding protein 80 (CBP80). In one embodiment, the compositions and methods inhibit the interaction between CBP80 and a member of the PGC1 family of co-activators. For example, in certain embodiments, the compositions and methods inhibit the interaction between CBP80 and human peroxisome proliferator-activated receptor gamma coactivator 1 beta (PGC1β) protein, proliferator-activated receptor gamma coactivator 1 alpha (PGC1α) protein, or PGC related coactivator (PRC).

In one aspect, the invention provides a CBP80, PRC, PGC1α or PGC1β-derived peptide. In one embodiment, the CBP80, PRC, PGC1α or PGC1β-derived peptide is a linear peptide. In some embodiments, the linear peptide comprises a sequence selected from SEQ ID NOs: 1-50. In another embodiment, the CBP80, PRC, PGC1α or PGC1β-derived peptide is a macrocyclic peptide. In some embodiments, the macrocyclic peptide has a structure of any one of Formula (I)-Formula (V).

In another aspect, the invention provides a method for inhibiting the interaction between CBP80 and the PGC1 family of co-activators. In one embodiment, the PGC1 family of co-activator is PRC, PGC1α or PGC1β. The invention also provides a method of treating or preventing cancer, heart disease, autoimmune disorders, obesity, diabetes, or chronic inflammation disorders. For example, in one embodiment, the invention provides method of treating or preventing a disease or disorder in a subject in need thereof by administering to the subject an effective amount of a composition of the invention. In specific embodiments, the invention provides a method of treating or preventing breast cancer. In some embodiments, the breast cancer is ER-negative breast cancer.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human. As used herein, a subject is preferably a mammal such as a non-primate (e.g., cows, pigs, horses, cats, dogs, rats, etc.) and a primate (e.g., monkey and human), most preferably a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “macrocycle” refers to a molecule having a chemical structure including a ring or cycle formed by at least 9 covalently bonded atoms.

The term “cyclic” and “macrocyclic” as used interchangeably herein, means having constituent atoms forming a ring. Thus, a “macrocyclic peptide-containing molecule” is a peptide-containing molecule that contains one or more rings (i.e., at least one ring) formed by atoms comprised in the molecule. “Cyclization” or “macrocyclization” as used herein refers to a process or reaction whereby a cyclic molecule is formed or is made to be formed. The term “peptidic backbone” as used herein refers to a sequence of atoms corresponding to the main backbone of a natural protein. A “non-peptidic backbone” as used herein refers to a sequence of atoms that does not correspond to a peptidic backbone.

As used herein, the terms “macrocyclic peptidomimetic” and “macrocyclic peptidomimetic molecule” refer to a compound comprising a plurality of amino acid residues joined by a plurality of peptide bonds and at least one macrocycle-forming linker which forms a macrocycle between the a carbon of one naturally-occurring amino acid residue or non-naturally-occurring amino acid residue or amino acid analog residue and the C-terminal carbonyl group (—C(O)—) of another naturally-occurring amino acid residue or non-naturally-occurring amino acid residue or amino acid analog residue. The macrocyclic peptidomimetics optionally include one or more (i.e., at least one) non-peptide bonds between one or more amino acid residues and/or amino acid analog residues, and optionally include one or more non-naturally-occurring amino acid residues or amino acid analog residues in addition to any which form the macrocycle.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “aromatic” refers to a carbocycle or heterocycle with one or more polyunsaturated rings and having aromatic character, i.e. having (4n+2) delocalized p (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “amide” or “amide group” employed alone or in combination with other terms, means, unless otherwise stated, a chemical group containing one or more amino groups. In one example, the amide group is represented by structure of —C(O)NRₐR_{b}, wherein the carbon atom may optionally be substituted with sulfate or phosphate atom.

As used herein, the term “attached to” refers to attaching two chemical groups through a chemical bond, for example a covalent bond or a non-covalent bond.

As used herein, the term “spacer” or “spacer unit” refers to a group which provides a physical linkage between a peptide of the invention and another entity such as a label molecule, an affinity tag, a small molecule, a biomolecule, a targeting agent, a solid support, a nanoparticle, and the like. The spacer is chosen so that it does not affect the biological properties of the peptide and so that it provides sufficient spacing between the peptide and the attached entity without compromising the biological properties of the peptides. In some embodiments, the spacer is a substituted alkyl, substituted alkoxy, substituted aryl, substituted heteroaryl, or substituted heteroalkyl group. Exemplary spaces include, but are not limited to, a C₂-C₁₂ alkyl group and a polyethylene glycol group (e.g., —(CH₂CH₂O)ₙ— where n=2-50).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates, in part, to the unexpected finding that the human cap-binding protein 80 (CBP80) binds to a short α-helical region that is conserved in human peroxisome proliferator-activated receptor gamma coactivator 1 alpha (PGC1α), human peroxisome proliferator-activated receptor gamma coactivator 1 beta (PGC1β), and PGC related coactivator (PRC). In one embodiment, the invention provides compositions for inhibiting the interaction between CBP80 and PGC1α, PGC1β, PRC, or each.

Structural analysis of the association complex between CBP80 and a PGC1β-derived peptide described herein revealed that the PGC1β-derived peptide adopts an alpha-helical conformation upon binding to CBP80. Based on the sequence similarity between the CBP80-binding motifs of PGC1β, PGC1α, and PRC, it is derived that PGC1α- and PRC-derived peptides also adopt an alpha-helical conformation upon binding to CBP80. Accordingly, in one aspect the invention provides an alpha-helical conformation of a PGC1β, PGC1α, or PRC-derived peptide. In one embodiment, the alpha-helical conformation of a PGC1β, PGC1α, or PRC-derived peptide is a macrocyclic/cyclic PGC1β, PGC1α, or PRC-derived peptide.

In one embodiment, the macrocyclic/cyclic peptides that are based on the sequence of the CBP80-binding domain in PGC1β (also referred to herein as “macrocyclic/cyclic PGC1β mimics”). In one embodiment, these peptides are cyclized via alkylation of two cysteines using a cross-linking agent, so that the overall distance of the resulting inter-side-chain linkage approximately matches the distance between the α-carbons of the corresponding residues in the CBP80-bound PGC1β -derived peptide. In another embodiment, these peptides are cyclized via a lactam bridge (e.g., via condensation of the side chain group of a lysine and glutamic acid reside). In another embodiment, these peptides are cyclized via a triazole-containing linker unit (e.g., via Huisgen cycloaddition of an alkyne- and an azide-containing amino acid). In another embodiment, these peptides are cyclized via a hydrocarbon linker unit (e.g., via ring-closing metathesis reaction between two alkyne-containing amino acids). In one embodiment, the overall distance of the resulting inter-side-chain linkage approximately matches the distance between the α-carbons of the corresponding residues in the CBP80-bound PGC1β-derived peptide.

In one embodiment, the invention provides a CBP80, PRC, PGC1α or PGC1β-derived peptide. In one embodiment, the peptide is a linear peptide comprising an amino acid sequence of any one of SEQ ID NOs: 1-50, or a fragment or variant thereof. In some embodiments, the invention provides a nucleic acid encoding a CBP80, PRC, PGC1α, or PGC1β-derived peptide. In one embodiment, the nucleic acid encodes a protein comprising an amino acid sequence of any one of SEQ ID NOs: 1-50, or a fragment or variant thereof. In some embodiments, the CBP80, PRC, PGC1α or PGC1β-derived peptide is a macrocyclic inhibitor. In one embodiment, the macrocyclic inhibitor links two amino acids within a peptide of any of SEQ ID NOs: 1-50 with a linker such that Cα carbon atoms connected by the linker unit is between about 10 and 18 Angstrom units. In one embodiment, the macrocyclic peptide is represented by any one of Formula (I)-Formula (V).

Further, it is contemplated herein that the novel peptides disclosed herein have various applications, including but not limited to cancer, heart disease, autoimmune disorders, obesity, diabetes, or chronic inflammation disorder applications. Accordingly, the invention provides methods for treating a disease or disorder in a subject in need thereof, including but not limited to cancer, heart disease, autoimmune disorders, obesity, diabetes, or chronic inflammation disorders. In one embodiment, the method comprises administering to the subject an effective amount of a composition comprising a peptide comprising an amino acid sequence of any one of SEQ ID NOs: 1-50, or a fragment or variant thereof. In another embodiment, the method comprises administering to the subject an effective amount of a nucleic acid encodes a protein comprising an amino acid sequence of any one of SEQ ID NOs: 1-50, or a fragment or variant thereof. In yet another embodiment, the method comprises administering to the subject an effective amount of a composition comprising a macrocyclic peptide represented by any one of Formula (I)-Formula (V).

### Compositions

In one aspect, the invention provides isolated peptides and compositions comprising isolated peptides. In one embodiment, the peptide inhibits the interaction between CBP80 and a binding partner. In one embodiment, the peptide binds to CBP80. The isolated peptides may be used, for example, to inhibit CBP80 binding to a binding partner. In one embodiment, the peptides inhibits CBP80 binding to a member of the PGC1 family of co-activators. In one embodiment, the peptide inhibits CBP80 binding to PGC1α, PGC1β, PRC or each.

In one embodiment, the peptide is a CBP80, PRC, PGC1α or PGC1β-derived peptide. In one embodiment, the peptide is a CBP80-derived peptide. In one embodiment, the CBP80-derived peptide is derived from PRC, PGC1α or PGC1β-binding domain of CBP80. In one embodiment, the CBP80-derived peptide mimics the PRC, PGC1α or PGC1β-binding domain of CBP80.

In one embodiment, the peptide is a PRC-derived peptide. In one embodiment, the PRC-derived peptide is derived from the CBP80-binding motif of PRC. In one embodiment, the PRC-derived peptide mimics the CBP80-binding motif of PRC.

In one embodiment, the peptide is a PGC1α-derived peptide. In one embodiment, the PGC1α-derived peptide is derived from the CBP80-binding motif in PGC1α. In one embodiment, the PGC1α-derived peptide mimics the CBP80-binding motif in PGC1α. In one embodiment, the PGC1α-derived peptide is derived from PGC1α amino acids 785-793.

In one embodiment, the peptide is a PGC1β-derived peptide. In one embodiment, the PGC1β-derived peptide is derived from the CBP80-binding motif in PGC1β. In one embodiment, the PGC1β-derived peptide mimics the CBP80-binding motif in PGC1β. In one embodiment, the PGC1β-derived peptide is derived from PGC1β amino acids 1011-1019.

In one aspect, the invention provides a linear peptide. In one embodiment, the linear peptide comprise a sequence selected from X₁X₂X₃X₄X₅X₆X₇X₈X₉X₁₀X₁₁X₁₂X₁₃X₁₄X₁₅X₁₆ (SEQ ID NO: 1) and X₂X₃X₄X₅X₆X₇X₈X₉X₁₀X₁₁X₁₂X₁₃X₁₄ (SEQ ID NO:2), wherein X₁ is Ala, Ser, or Thr; X₂ is Met, Leu, Ala, Ile, or Val; X₃ is Asp, Ala, Glu, Asn, Gln, Ser, or Thr; X₄ is Phe or Ala; X₅ is Asp, Glu or Ala; X₆ is Ser, Thr, Ala, Glu, Asp, Gln, Asn, Phe, Tyr, Trp, Met, Leu, Ile, Val, Lys, His, Arg, or Aib; X₇ is Leu, Ile or Ala; X₈ is Leu, Ile or Ala; X₉ is Lys, Ala, Ser, Thr, Glu, Asp, Gln, Asn, Phe, Tyr, Trp, Met, Leu, Ile, Val, His, Arg, Aib, a lysine derivative, a omithine derivative, or a 2,4-diaminobutyric acid derivative; X₁₀ is Glu, Gln, Ala, Ser, Thr, Asp, Asn, Phe, Tyr, Trp, Met, Leu, Ile, Val, Lys, His, Arg, or Aib; X₁₁ is Ala, Gly, Leu, Ile, or Aib; X₁₂ is Gln, Ala, Asn, Ser, Thr, Glu, Asp, Asn, Phe, Tyr, Trp, Met, Leu, Ile, Val, Lys, His, Arg, or Aib; X₁₃ is Gln, Arg, Lys, Ala, Ser, Thr, Glu, Asp, Asn, Phe, Tyr, Trp, Met, Leu, Ile, Val, or His; X₁₄ is Ser, Asn, Ala, Thr, Glu, Asp, Gln, Phe, Tyr, Trp, Met, Leu, Ile, Val, Lys, His, or Arg; X₁₅ is Leu; and X₁₆ is His, Arg.

In some embodiments, X₉ is a lysine, omithine or 2,4-diaminobutyric acid derivative bearing a side-chain electrophilic group capable of reacting with cysteine. In one embodiment, the electrophilic group capable of reacting with cysteine is selected from the group consisting of

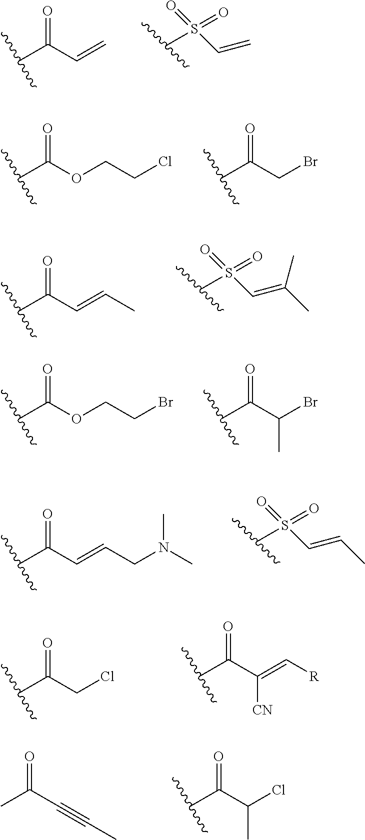

wherein R is a C₃-C₂₀ alkyl, C₃-C₂₀ heteroalkyl, C₆-C₁₀ aryl, or C₆-C₁₀ heteroaryl group.

In some embodiments, the peptide comprises a group on the N-terminus. Exemplary N-terminal groups include, but is not limited to, a hydrogen, an acetyl, and a label.

In one embodiment, the label is selected from a group including, but not limited to, an affinity label molecule, a photoaffinity label, a dye, a chromophore, a fluorescent molecule, a phosphorescent molecule, a chemiluminascent molecule, an energy transfer agent, a photocrosslinker molecule, a redox-active molecule, an isotopic label molecule, a spin label molecule, a metal chelator, a metal-comprising moiety, a heavy atom-comprising-moiety, a radioactive moiety, a contrast agent molecule, a MRI contrast agent, an isotopically labeled molecule, a PET agent, a polypeptide, a cell penetrating polypeptide, a carbohydrate, a polynucleotide, a peptide nucleic acid, a fatty acid, a lipid, biotin, a biotin analogue, a polymer, a small molecule, a drug or drug candidate, a cytotoxic molecule, a solid support, a surface, a resin, a nanoparticle, a quantum dot and any combination thereof.

In some embodiments, the peptide comprises a group on the C-terminus. Exemplary C-terminal groups includes, but is not limited to a free carboxylic group, an amide group, or a cell penetrating peptide. In one embodiment, the cell penetrating peptide is attached to the C-terminus of the peptide via a spacer.

In one embodiment, the spacer is a substituted alkyl, substituted alkoxy, substituted aryl, substituted heteroaryl, or substituted heteroalkyl group. In another embodiment, the spacer is a C₂-C₁₂ alkyl group or a polyethylene glycol group. In exemplary polyethylene glycol group include, but are not limited to —(CH₂CH₂O)ₙ— where n=2-50.

In one embodiment, the linear peptide comprises a sequence selected from any one of SEQ ID NO:1 to SEQ ID NO:50.

TABLE 1
Peptides
Sequence

X₁X₂X₃X₄X₅X₆X₇X₈X₉X₁₀X₁₁X₁₂X₁₃X₁₄X₁₅X₁₆ (SEQ ID NO: 1)
X₂X₃X₄X₅X₆X₇X₈X₉X₁₀X₁₁X₁₂X₁₃X₁₄ (SEQ ID NO: 2)
SGG-AMDFDSLLKEAQQSLH (SEQ ID NO: 3)
AMDFDSLLKEAQQSLH (SEQ ID NO: 4)
SLDFDSLLKEAQRSLRR (SEQ ID NO: 5)
SLDFDDLLKQAQKNLRR (SEQ ID NO: 6)
MDFDSLLKEAQQS (SEQ ID NO: 7)
MAFDSLLKEAQQS (SEQ ID NO: 8)
MDADSLLKEAQQS (SEQ ID NO: 9)
MDFASLLKEAQQS (SEQ ID NO: 10)
MDFDALLKEAQQS (SEQ ID NO: 11)
MDFDSALKEAQQS (SEQ ID NO: 12)
MDFDSLAKEAQQS (SEQ ID NO: 13)
MDFDSLLAEAQQS (SEQ ID NO: 14)
MDFDSLLKAAQQS (SEQ ID NO: 15)
MDFDSLLKEGQQS (SEQ ID NO: 16)
MDFDSLLKEAAQS (SEQ ID NO: 17)
MDFDSLLKEAQAS (SEQ ID NO: 18)
MAFDSLLKNAAAS (SEQ ID NO: 19)
MAFDSLLKNAAQS (SEQ ID NO: 20)
MAFDSLLKAAQQS (SEQ ID NO: 21)
MAFDSLLK(Aib)AQQS (SEQ ID NO: 22)
MAFDSMLKAAQQS (SEQ ID NO: 23)
MDFDSLL(CrtDab)EAQQS (SEQ ID NO: 24)
MDFDSLLKEAQQSRRRRRRRR (SEQ ID NO: 25)
Biot-Ahx-SGG-AMDFDSLLKEAQQSLH-NH₂ (SEQ ID NO: 26)
Ac-AMDFDSLLKEAQQSLH-NH₂ (SEQ ID NO: 27)
Ac-SLDFDSLLKEAQRSLRR-NH₂ (SEQ ID NO: 28)
Ac-SLDFDDLLKQAQKNLRR-NH₂ (SEQ ID NO: 29)
Ac-MDFDSLLKEAQQS-NH₂ (SEQ ID NO: 30)
Ac-MAFDSLLKEAQQS-NH₂ (SEQ ID NO: 31)
Ac-MDADSLLKEAQQS-NH₂ (SEQ ID NO: 32)
Ac-MDFASLLKEAQQS-NH₂ (SEQ ID NO: 33)
Ac-MDFDALLKEAQQS-NH₂ (SEQ ID NO: 34)
Ac-MDFDSALKEAQQS-NH₂ (SEQ ID NO: 35)
Ac-MDFDSLAKEAQQS-NH₂ (SEQ ID NO: 36)
Ac-MDFDSLLAEAQQS-NH₂ (SEQ ID NO: 37)
Ac-MDFDSLLKAAQQS-NH₂ (SEQ ID NO: 38)
Ac-MDFDSLLKEGQQS-NH₂ (SEQ ID NO: 29)
Ac-MDFDSLLKEAAQS-NH₂ (SEQ ID NO: 40)
Ac-MDFDSLLKEAQAS-NH₂ (SEQ ID NO: 41)
Ac-MAFDSLLKNAAAS-NH₂ (SEQ ID NO: 42)
Ac-MAFDSLLKNAAQS-NH₂ (SEQ ID NO: 43)
Ac-MAFDSLLKAAQQS-NH₂ (SEQ ID NO: 44)
Ac-MAFDSLLK(Aib)AQQS-NH₂ (SEQ ID NO: 45)
Ac-MAFDSMLKAAQQS-NH₂ (SEQ ID NO: 46)
Ac-MDFDSLL(CrtDab)EAQQS-NH₂ (SEQ ID NO: 47)
Ac-MDFDSLLKEAQQSRRRRRRRR-NH₂ (SEQ ID NO: 48)

In another aspect, the invention provides a macrocyclic peptide. In one embodiment, the macrocyclic peptide is represented by any one of Formula (I) to Formula (V):

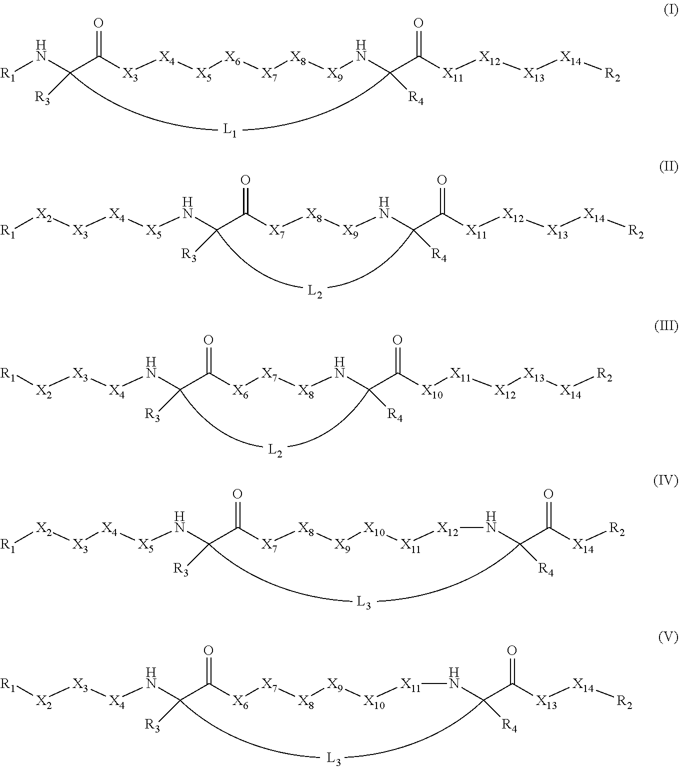

wherein X₂ is Met, Leu, Ala, Ile, or Val; X₃ is Asp, Ala, Glu, Asn, Gln, Ser, or Thr; X₄ is Phe or Ala; X₅ is Asp, Glu or Ala; X₆ is Ser, Thr, Ala, Glu, Asp, Gln, Asn, Phe, Tyr, Trp, Met, Leu, Ile, Val, Lys, His, Arg, or Aib; X₇ is Leu, Ile or Ala; X₈ is Leu, Ile or Ala; X₉ is Lys, Ala, Ser, Thr, Glu, Asp, Gln, Asn, Phe, Tyr, Trp, Met, Leu, Ile, Val, His, Arg, Aib, a lysine derivative, a omithine derivative, or a 2,4-diaminobutyric acid derivative; X₁₀ is Glu, Gln, Ala, Ser, Thr, Asp, Asn, Phe, Tyr, Trp, Met, Leu, Ile, Val, Lys, His, Arg, or Aib; X₁₁ is Ala, Gly, Leu, Ile, or Aib; X₁₂ is Gln, Ala, Asn, Ser, Thr, Glu, Asp, Asn, Phe, Tyr, Trp, Met, Leu, Ile, Val, Lys, His, Arg, or Aib; X₁₃ is Gln, Arg, Lys, Ala, Ser, Thr, Glu, Asp, Asn, Phe, Tyr, Trp, Met, Leu, Ile, Val, or His; X₁₄ is Ser, Asn, Ala, Thr, Glu, Asp, Gln, Phe, Tyr, Trp, Met, Leu, Ile, Val, Lys, His, or Arg;

R₁ is hydrogen, an acetyl group, or a label molecule, wherein the label molecule optionally comprises a spacer unit;

R₂ is a free carboxylic group or an amide group;

R₃ is hydrogen or methyl group;

R₄ is hydrogen or methyl group;

L₁ is a linker unit, such that the linear dimension between the Cα carbon atoms connected by the linker unit is between about 10 and 18 Angstrom units;

L₂ is —(CH₂)₃CH═CH(CH₂)₃— or —(CH₂)₈—; and

L₃ is —(CH₂)₆CH═CH(CH₂)₃— or —(CH₂)₁₁—.

In some embodiments, the linker unit L₁ includes, but is not limited to, —CH₂S(CH₂)ₙSCH₂—, wherein n is an integer number comprised between 4 and 8;

—CH₂SCH₂CH═CHCH₂SCH₂—;

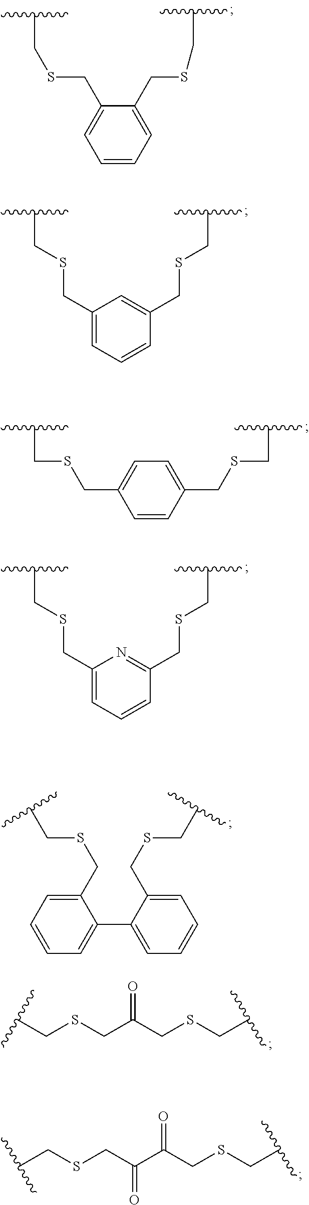

—(CH₂)ₘNHCO(CH₂)ₙ—, wherein m is an integer number between 2 and 4, and n is an integer number between 1 and 2;

—(CH₂)ₙCONH(CH₂)ₘ—, wherein m is an integer number between 2 and 4, and n is an integer number between 1 and 2;

—(CH₂)ₘCH═CH(CH₂)ₙ—, wherein m is an integer number between 1 and 6, and n is an integer number between 1 and 6;

—(CH₂)ₘCH₂CH₂(CH₂)ₙ—, wherein m is an integer number between 1 and 6, and n is an integer number between 1 and 6;

—(CH₂)ₘC≡C(CH₂)ₙ—, wherein m is an integer number between 1 and 6, and n is an integer number between 1 and 6; and

—(CH₂)ₘ(triazole)(CH₂)ₙ—, wherein m is an integer number between 1 and 6, and n is an integer number between 1 and 6.

In some embodiments, X₉ is a lysine, ornithine or 2,4-diaminobutyric acid derivative bearing a side-chain electrophilic group capable of reacting with cysteine. In one embodiment, the electrophilic group capable of reacting with cysteine is selected from the group consisting of

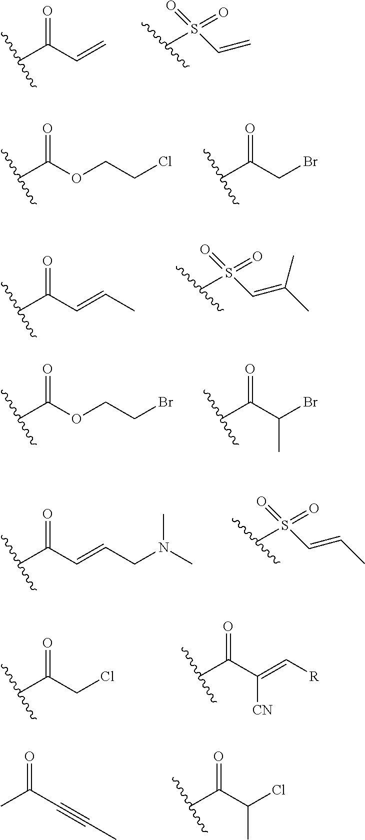

wherein R is a C₃-C₂₀ alkyl, C₃-C₂₀ heteroalkyl, C₆-C₁₀ aryl, or C₆-C₁₀ heteroaryl group.

In some embodiments, the macrocyclic/cyclic peptide includes, but is not limited to,

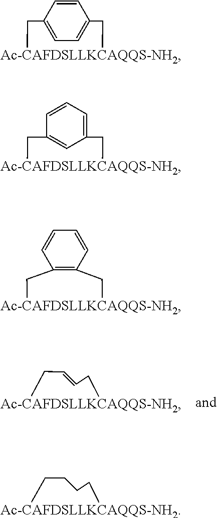

and

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer. Representative methods for preparing the peptides of the invention are provided in Example 4-6.

The invention should also be construed to include any form of a peptide having substantial homology to a peptide disclosed herein. Preferably, a peptide which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a peptide disclosed herein.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of a peptide of the invention.

A peptide or protein of the invention may be phosphorylated using conventional methods such as the method described in Reedijk et al. (The EMBO Journal 11(4):1365, 1992).

Cyclic derivatives of the peptides of the invention are also part of the present invention. Cyclization may allow the peptide to assume a more favorable conformation for association with other molecules. For example, cyclization may allow the peptide to assume a more favorable conformation for association with a target protein. Accordingly, cyclization may result in improved binding affinity and specificity toward the target protein. Cyclization may also confer to the peptide beneficial properties such as increased resistance against proteolysis, increased cell permeability, and/or more favorable pharmacokinetic properties such as oral bioavailability, reduced clearance, and the like. In one embodiment, the cyclization of a peptide of the invention stabilizes the peptide into an α-helical conformation.

Cyclization may be achieved using techniques known in the art. These methods include the use of covalent inter-side-chain linkages such as disulfide bonds (Jackson, King et al. 1991), lactam (Osapay and Taylor 1992), thioether (Brunel and Dawson 2005) or triazole (Scrima, Le Chevalier-Isaad et al. 2010; Kawamoto, Coleska et al. 2012) bridges, ‘hydrocarbon staples’ (Blackwell and Grubbs 1998; Schafmeister, Po et al. 2000; Bemal, Wade et al. 2010), and cysteine cross-linking moieties (Zhang, Sadovski et al. 2007; Muppidi, Wang et al. 2011; Jo, Meinhardt et al. 2012; Spokoyny, Zou et al. 2013). Another known approach for stabilization of α-helical peptides involves the introduction of so-called ‘hydrogen bond surrogates’, i.e. hydrocarbon linkages replacing an N-terminal i/i+4 hydrogen bond (Wang, Liao et al. 2005). Any of these methods, or combination thereof, can be applied to stabilize the α-helical conformation of PGC1β-, PGC1α-, or PRC-derived peptides for the purpose of developing inhibitors of the interaction between CBP80 and members of the PGC1 family of co-activators.

Other methods of cyclization disulfide bonds which may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to peptides comprising a peptide fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue (e.g., bone, regenerating bone, degenerating bone, cartilage). A targeting domain may target the peptide of the invention to a cellular component.

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the peptide fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Nucleic Acid

In one embodiment, the present invention provides isolated nucleic acids and compositions comprising isolated nucleic acids encoding a CBP80, PRC, PGC1α or PGC1β-derived peptide of the invention. In one embodiment, the isolated nucleic acid encodes a peptide having a sequence of one of SEQ ID NO: 1 to SEQ ID NO:50

Further, the invention encompasses an isolated nucleic acid encoding a peptide having substantial homology to a peptide disclosed herein. In certain embodiments, the isolated nucleic acid sequence encodes a peptide having at least 75%, 80%, 85%, 90%, 95%, 96%, 97%, 98%, or 99% sequence homology with an amino acid sequence selected from SEQ NOs: 1-25.

The isolated nucleic acid sequence encoding the peptide can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding a CBP80, PRC, PGC1α or PGC1β-derived peptide, or functional fragment thereof. In one embodiment, the composition comprises an isolated RNA molecule encoding a CBP80, PRC, PGC1α or PGC1β-derived peptide, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding a CBP80, PRC, PGC1α or PGC1β-derived peptide is typically achieved by operably linking a nucleic acid encoding the CBP80, PRC, PGC1α or PGC1β-derived peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a CBP80, PRC, PGC1α or PGC1β-derived peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Delivery System

In one embodiment, the present invention provides a delivery vehicle comprising a CBP80, PRC, PGC1α or PGC1β-derived peptide, or a nucleic acid molecule encoding a CBP80, PRC, PGC1α or PGC1β-derived peptide. Exemplary delivery vehicles include, but are not limited to, microspheres, microparticles, nanoparticles, polymerosomes, liposomes, and micelles. For example, in certain embodiments, the delivery vehicle is loaded with a CBP80, PRC, PGC1α or PGC1β-derived peptide, or a nucleic acid molecule encoding a CBP80, PRC, PGC1α or PGC1β-derived peptide. In certain embodiments, the delivery vehicle provides for controlled release, delayed release, or continual release of its loaded cargo. In certain embodiments, the delivery vehicle comprises a targeting moiety that targets the delivery vehicle to a treatment site.

The present invention provides a scaffold or substrate composition comprising a CBP80, PRC, PGC1α or PGC1β-derived peptide, a nucleic acid molecule encoding a CBP80, PRC, PGC1α or PGC1β-derived peptide, a cell producing a CBP80, PRC, PGC1α or PGC1β-derived peptide, or a combination thereof. For example, in one embodiment, a CBP80, PRC, PGC1α or PGC1β-derived peptide, a nucleic acid molecule encoding a CBP80, PRC, PGC1α or PGC1β-derived peptide, a cell producing a CBP80, PRC, PGC1α or PGC1β-derived peptide, or a combination thereof within a scaffold. In another embodiment CBP80, PRC, PGC1α or PGC1β-derived peptide, a nucleic acid molecule encoding a CBP80, PRC, PGC1α or PGC1β-derived peptide, a cell producing a CBP80, PRC, PGC1α or PGC1β -derived peptide, or a combination thereof is applied to the surface of a scaffold. The scaffold of the invention may be of any type known in the art. Non-limiting examples of such a scaffold includes a, hydrogel, electrospun scaffold, foam, mesh, sheet, patch, and sponge.

The present invention also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this invention may be carried out, for example, by parenteral, by intravenous, intratumoral, subcutaneous, intramuscular, or intraperitoneal injection, or by infusion or by any other acceptable systemic method.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

### Methods

In one aspect, the invention provides methods to inhibit the interaction between CBP80 and a member of the PGC1 family of co-activators. In one embodiment, the method comprises administering an effective amount of a CBP80, PRC, PGC1α or PGC1β-derived peptide to an individual in need of inhibition of the interaction between CBP80 and a PGC1 family of co-activator. In one embodiment, the member of the PGC1 family of co-activators is PRC, PGC1α or PGC1β.

The invention also provides methods for the treatment or prevention of a disease or disorder in a subject in need thereof. Exemplary diseases and disorders treated or prevented by way of the present invention, include, but are not limited, to cancer, heart disease, autoimmune disorders, obesity, diabetes, and chronic inflammation disorders. In some embodiments, the method comprises administering an effective amount of a composition described herein to a subject diagnosed with, suspected of having, or at risk for developing a condition associated with cancer, heart disease, autoimmune disorders, obesity, diabetes, or chronic inflammation disorders.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

In one embodiment, the method comprises administering to the subject a composition comprising a peptide comprising a sequence of any if SEQ ID NO: 1 to SEQ ID NO:50. In one embodiment, the method comprises administering to the subject a composition comprising a macrocyclic peptide represented by any of Formula (I) to Formula (V).

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are preferably administered by i.v. injection.

In one embodiment, the invention provides methods of treating or preventing heart disease, autoimmune disorders, obesity, diabetes, or chronic inflammation disorders. In some embodiments, the method comprises administering to a subject an effective amount of a linear or macrocyclic peptide of the invention.

PGC1β has been shown to be important in the activation of macrophages by interferon (IFN)-gamma. IFN-gamma activation is important to the immune system and as part of auto-immune disease pathogenesis. PRC has been shown to regulate a number of inflammation and stress genes and specifically as a result of mitochondrial stress. Accordingly, in one aspect, the invention provides methods of treating or preventing an autoimmune or inflammatory disease or disorder.

In some embodiments of the methods for treating or preventing an autoimmune or inflammatory disease or disorder in subject in need thereof, a second agent is administered to the subject, such as an immunomodulatory agent.

Exemplary immunomodulatory agents include, but are not limited to, a cytokine, a stem cell growth factor, a lymphotoxin, a hematopoietic factor, a colony stimulating factor (CSF), an interferon (IFN), erythropoietin, lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β or -γ, and stem cell growth factor, such as that designated “S1 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and LT. In one embodiment, the second therapeutic cis interferon-gamma.

The following are non-limiting examples of autoimmune or inflammatory disease or disorder that can be treated by the disclosed methods and compositions: acquired immunodeficiency disease syndrome (AIDS), autoimmune lymphoproliferative syndrome, hemolytic anemia, inflammatory diseases, and thrombocytopenia, acute or chronic immune disease associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis. Lyme arthritis, psoriatic arthritis and reactive arthritis), autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycaemia, autoimmune neutropaenia, autoimmune thrombocytopaenia, autoimmune thyroid disease, B cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinaemia), conjunctivitis, connective tissue disease associated interstitial lung disease, contact dermatitis, Coombs positive haemolytic anaemia, cor pulmonale, Creutzfeldt-Jakob disease, cryptogenic autoimmune hepatitis, cryptogenic fibrosing alveolitis, culture negative sepsis, cystic fibrosis, cytokine therapy associated disorders, Crohn's disease, dementia pugilistica, demyelinating diseases, dengue hemorrhagic fever, dermatitis, dermatitis scleroderma, dermatologic conditions, dermatomyositis/polymyositis associated lung disease, diabetes, diabetic arteriosclerotic disease, diabetes mellitus, Diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine, receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram negative sepsis, gram positive sepsis, granulomas due to intracellular organisms, group B streptococci (GBS) infection, Grave's disease, haemosiderosis associated lung disease, hairy cell leukemia, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpurea, Hepatitis A, Hepatitis B. Hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopaenia, idiopathic pulmonary fibrosis, idiopathic thrombocytopaenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anaemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease associated lung disease, monoclonal gammopathy, multiple myeloma, multiple systems degenerations (Mencel Dejerine-Thomas Shi-Drager and Machado-Joseph), myalgic encephalitis/Royal Free Disease, myasthenia gravis, microscopic vasculitis of the kidneys, mycobacterium avium intracellulare, mycobacterium tuberculosis, myclodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, Non-alcoholic Steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaccus, pemphigoid, perennial rhinitis, pericardial disease, peripheral atherlosclerotic disease, peripheral vascular disorders, peritonitis, pernicious anemia, phacogenic uveitis, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, Progressive supranucleo Palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis associated interstitial lung disease, rheumatoid spondylitis, sarcoidosis. Schmidt's syndrome, scleroderma, senile chorea, Senile Dementia of Lewy body type, sepsis syndrome, septic shock, seronegative arthropathies, shock, sickle cell anemia, Sjogren's disease associated lung disease, Sjorgren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, spondyloarthopathy, sporadic, polyglandular deficiency type I sporadic, polyglandular deficiency type II, Still's disease, streptococcal myositis, stroke, structural lesions of the cerebellum, Subacute sclerosing panencephalitis, sympathetic ophthalmia, Syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, systemic sclerosis, systemic sclerosis-associated interstitial lung disease, T-cell or FAB ALL, Takayasu's disease/arteritis, Telangiectasia, Th2 Type and Th1 Type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wemicke-Korsakoff syndrome. Wilson's disease, xenograft rejection of any organ or tissue, yersinia and salmonella-associated arthropathy.

PGC-1α knockout mice are incredibly lean and resistant to diet-induced obesity. Further, PGC-1α has been linked to glucose intolerance, insulin resistance, and diabetes, but it is unknown whether it is protective or a mediator of disease progression. Polymorphisms in PGC-1α have been shown to either increase or not be associated with risk of diabetes depending on the study. PGC1-α activity has been shown to be increased in the diabetic liver and pancreas. Increased PGC1-α activity stimulates glucose production (increases blood glucose levels), suppresses insulin secretion, but also reduces oxidative phosphorylation. Accordingly, in one aspect, the invention provides methods of treating or preventing obesity or diabetes.

In some embodiments of the methods for treating or preventing obesity or diabetes subject in need thereof, a second agent is administered to the subject, such as an anti-diabetic agent. The method of the present invention may be combined with additional treatment or treatments.

Examples of the known antidiabetic agent include PPAR-α a agonists, PPAR-δ agonists, retinoid RXR agonists, β3-adrenaline receptor agonists, 11β-hydroxysteroid dehydrogenase inhibitors, protein tyrosine phosphatase-1B (PTP-1B) inhibitors, AMP-activated protein kinase (AMPK) activators, acetyl-CoA carboxylase (ACC) inhibitors, cannabinoid receptor 1 (CB1) antagonists, insulin secretagogues (ATP-dependent potassium channel inhibitors (sulfonylurea drugs, sulfonamide drugs, phenylalanine derivatives and the like)), biguanides, α-glucosidase inhibitors, insulin formulations, insulin analogues, dipeptidyl peptidase IV inhibitors, glucagon-like peptide 1 (GLP1) agonists and GLP1. These known antidiabetic agents may also be administered in the form of a combination drug. Alternatively, a plurality of individual agents may be administered at the same time. Alternatively, the individual agents may be administered at appropriate intervals.

PGC-1 proteins are important for metabolic changes (such as to the hypoxic environment of a tumor), angiogenesis, metastasis, and integration of many different signaling pathways (in constitutively active growth-signaling or in evasion of apoptotic signaling), and have been shown to modulate effectiveness of existing chemotherapeutics. Accordingly, in one aspect, the invention provides methods of treating or preventing cancer, or of treating and preventing metastasis of tumors. For example, it is described herein that the interaction of PGC1β with CBP80 alters human gene expression in ER-positive breast cancer cells that are responsive to ER and ERR and in ER-negative breast cancer cells that are responsive to ERR provides. Accordingly, in one embodiment, the linear and macrocyclic peptides described herein can thus be used to treat breast cancer. In one embodiment, the linear and macrocyclic peptides described herein can thus be used to treat ER-negative breast cancer.

In one embodiment, the invention provides a method of treating or preventing cancer in a subject in need thereof, the method comprising administering to the subject an effective amount of a composition comprising a linear peptide selected from SEQ ID NO:1 to SEQ ID NO:50, a fragment thereof, variant thereof or a nucleic acid sequence encoding SEQ ID NO:1 to SEQ ID NO:50, a fragment thereof, variant thereof. In one embodiment, the invention provides a method of treating or preventing cancer in a subject in need thereof, the method comprising administering to the subject an effective amount of a composition comprising a macrocyclic peptide represented by one of Formula (I)-Formula (V).

In some embodiments of the methods for treating or preventing cancer in a subject in need thereof, a second agent is administered to the subject, such as an antineoplastic agent or a chemotherapeutic agent.

In another embodiment, the invention provides a method to treat cancer comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compositions of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

The following are non-limiting examples of cancers that can be treated by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

### Pharmaceutical Compositions

The present invention envisions treating a disease, for example, cancer, heart disease, autoimmune disorders, obesity, diabetes, or chronic inflammation disorders and the like, in a subject by the administration of a CBP80, PRC, PGC1α or PGC1β-derived peptide or variant thereof or a nucleic acid sequence encoding a CBP80, PRC, PGC1α or PGC1β-derived peptide or variant thereof.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent or modified cell may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The agents of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, are not to be construed as limiting in any way the remainder of the disclosure.

### Example 1: The PGC1β C-Terminus Binds CBP80 In Vitro

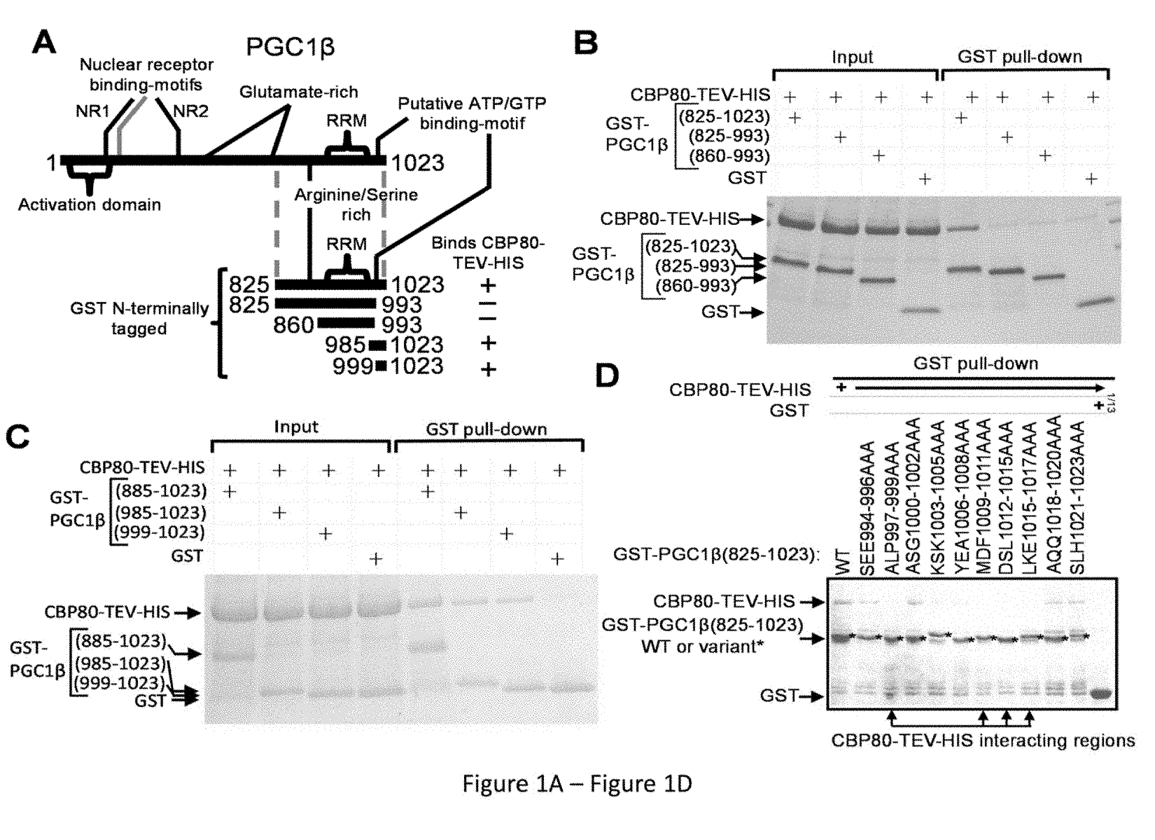

Yeast-2 hybrid results (Kataoka et al., 1995, Nucleic Acids Res 23:3638) indicated that CBP80 interacts with PGC1β C-terminal to its glutamate rich region (FIG. 1A). Using GST pull-downs of GST-fused PGC1β protein regions and mutated variants thereof, it was observed that: the interaction with CBP80-HIS (baculovirus-produced) is precluded when C-terminal amino acids 994-1023 are absent, 994-1023 fused to GST bind CBP80 alone, and CBP80-HIS interacts with GST-PGC1β amino acid regions 998-999 and 1009-1017 (FIGS. 1B-D). These interacting regions are C-terminal to the RRM and together form a predicted C-terminal α-helix with an ATP/GTP-binding loop region that connects to the RNA-recognition motif (RRM); since PGC1β functions in mitochondrial respiration, this suggests that an ATP-sensing allosteric regulator might control the association of RNA and/or CBP80 with PGC1β.

### Example 2: 2.7 Å-Resolution X-Ray Crystal Structure of CBP80-CBP20 (CBC) Bound to the Helix of PGC1β Via CBP80

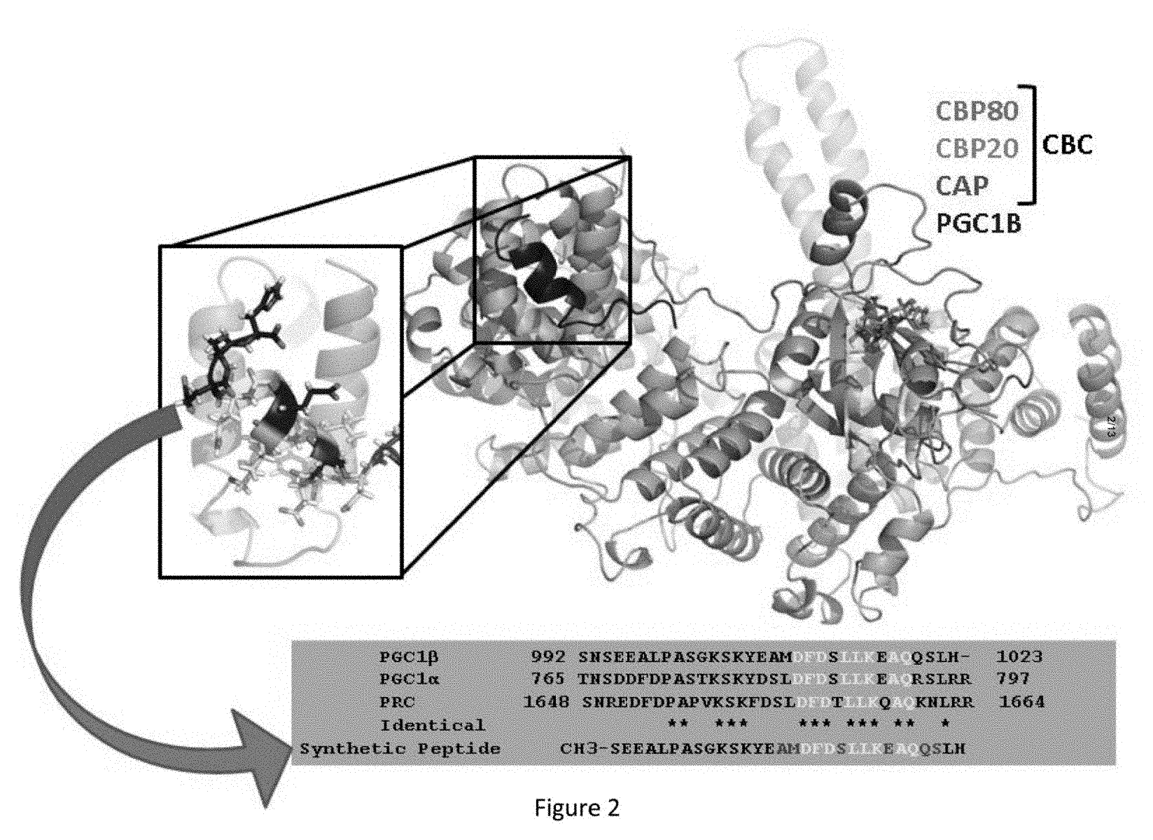

Experiments were conducted to collect X-ray diffraction data from a very large (900 μm long) crystal of the PGC1β-CBC-cap complex and, using prior CBC and CBC-cap structures, (Calero et al., 2002, Nat Struct Biol 9:912; Mazza et al., 2002, EMBO J 21:4458; Mazza et al., 2001, Mol Cell 8:383; Mazza et al., 2002, Acta Crystallogr D Biol Crystallogr 58:2194) have phased through molecular replacement and refined the structure to 0.21/0.27 R_{Model}/R_{Free}. By so doing, it was discovered that extra electron density corresponding to an α-helix matched the side-chain residues of the PGC1β C-terminal helix. Notably, the position of the α-helix does not interfere with CBP20 or importin-α (Dias et al., 2009, 16:930; Sato et al., 2009, Genes Dev 23:2537) binding positions on CBP80. CBP80-facing residues of PGC1β are identical in all PGC1 paralogs (FIG. 2), suggesting that the latter may bind CBP80 in a similar α-helical conformation.

### Example 3: Macrocyclic Inhibitors of the Human PGC1β-CBP80 Protein-Protein Interaction

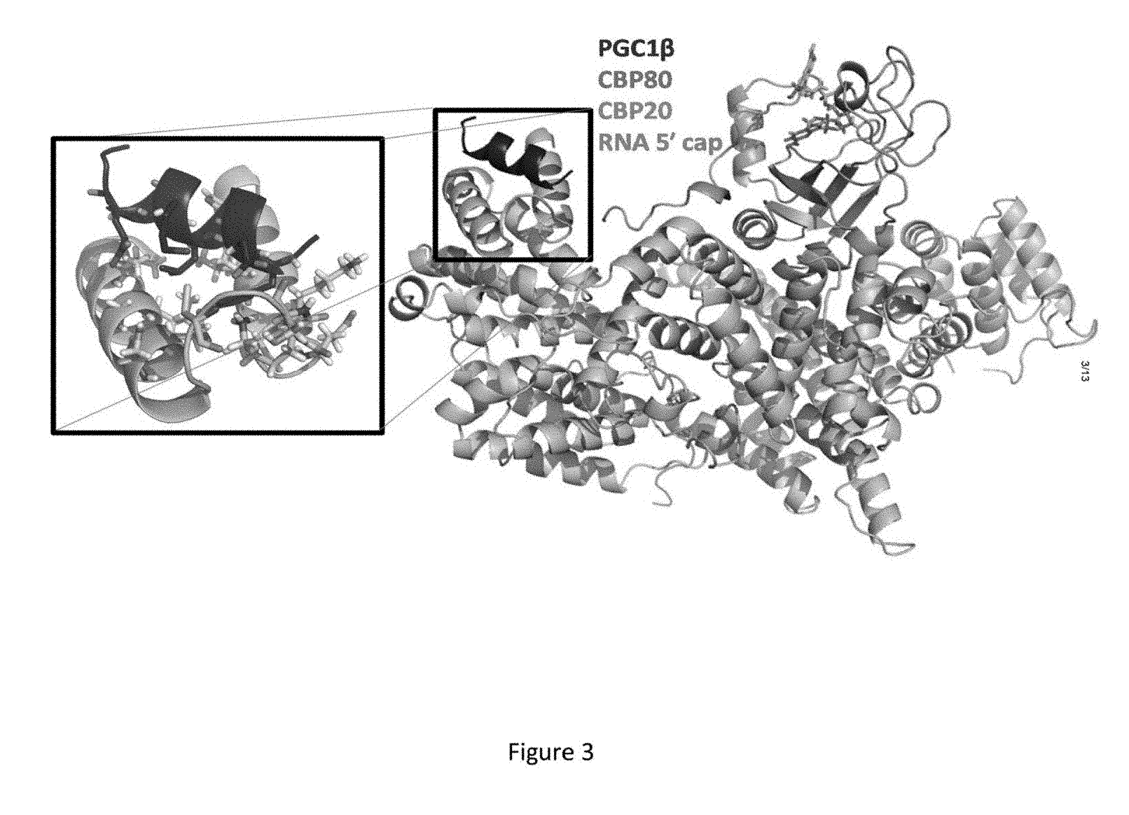

The experiments were designed to combine novel technologies to generate peptidomimetic inhibitors of α-helix mediated protein-protein interactions, with the structural characterization of a novel protein-protein interaction involving the human cap-binding protein 80 (CBP80) and a short α-helical region from the human peroxisome proliferator-activated receptor gamma coactivator 1 beta (PGC1β) protein (FIG. 3). PGC1β binds the estrogen receptor (ER) and promotes the growth of ER-positive breast cancers. PGC1β also revs up the metabolism of ER-negative breast cancer cells by binding ER-related receptor (ERR). This project aims to develop cell-permeable PGC1β peptidomimetics which interfere with PGC1β function in cells and reduce the viability of breast cancer cells. By blocking the interaction of PGC1β with CBP80, inhibition of the expression of genes that are responsive to ER and ERR in ER-positive breast cancer cells and those responsive to ERR in ER-negative breast cancer cells is expected. This mechanism of action would differ from that of therapeutic agents currently in use for treating ER-positive breast cancer and would provide an entirely new strategy for targeting ER-negative breast cancer.

Experiments were designed for the purpose of testing and validating the α-helix peptidomimetic technology as a new drug discovery platform for ‘undruggable’ targets such as protein-protein interactions.

The structure of the PGC1β-CBP80 complex provides a strong competitive edge over any other group potentially interested in targeting this protein-protein interaction. Being mediated by an α-helical domain, this protein-protein interaction is perfectly suited for targeting by the α-MOrPH technology. Previous studies with HDM2 antagonists show that α-MOrPHs are more cell permeable than ‘hydrocarbon-stapled peptides’, which constitute the major competing α-helix mimicry technology available to date. Finally, targeting the PGC1β-CBP80 interaction is expected to provide an entirely new approach to minimize the expression of ER- and ERR-regulated genes.

### Synthesize and Assess Inhibitory Activities of a MOrPHs In Vitro

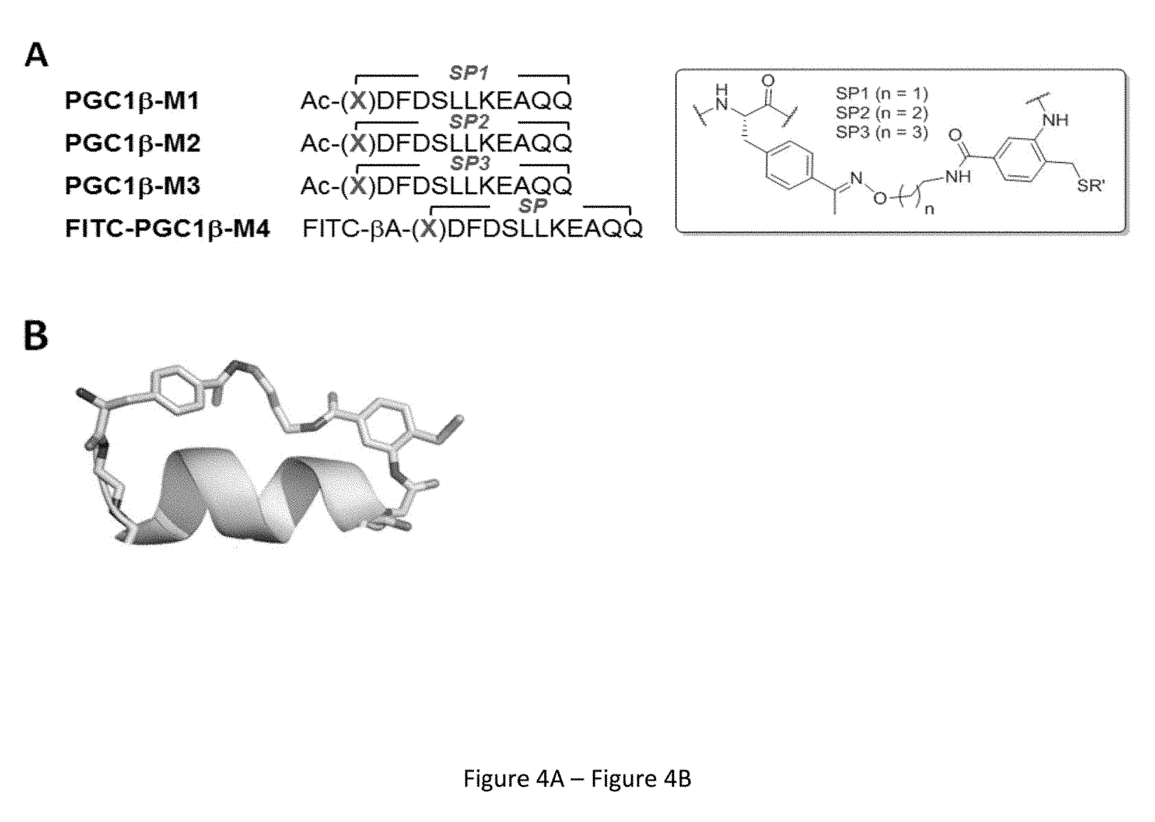

The X-ray crystal structure of a portion of PGC1β bound to the CBP80-CBP20-cap complex is depicted in FIG. 3, providing first-time insight into a viable molecular interface for interfering with cellular PGC1β activity. In particular, structural analyses revealed that PGC1β interacts via a short α-helical region with CBP80. Based on these and other data, a first-generation of PGC1β peptidomimetics were designed to inhibit the PGC1β-CBP80 interaction following the principles defined by the Fasan group for the creation of macrocyclic α-helix peptidomimetics (Smith et al., 2014 Chemm Commun 50:5027; Fasan et al. PCT/US15/23883; FIG. 4).

These inhibitory “α-MOrPHs” comprise the α-helical CBP80-binding domain of PGC1β constrained by a non-peptidic linker that connects the α-sidechain of the peptide to its C-terminus.

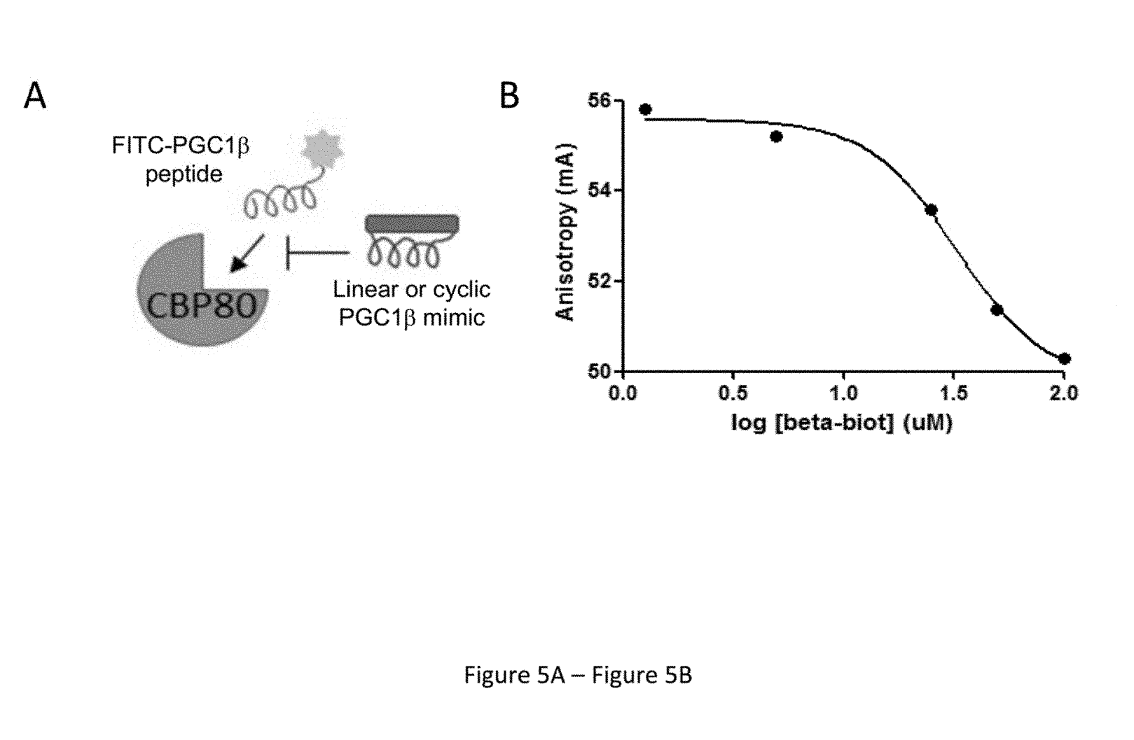

The ability of α-MOrPHs to disrupt the PGC1β-CBP80 interaction will be assessed and quantified via a fluorescence polarization-based competition assay, in which disruption of the complex formed between a fluorescein-conjugated PGC1β peptide and CBP80 is measured upon adding increasing concentrations of each inhibitory α-MOrPH (FIG. 5A). Using this assay, it is established herein that a linear peptide spanning the CBP80-binding domain of PGC1β (which is functionally analogous to what in the future will be an α-MOrPH; FIG. 5A) can effectively disrupt the interaction between a FITC-labeled PGC1β peptide and CBP80 (IC₅₀=31 μM, FIG. 3B), thus demonstrating the feasibility of targeting this protein-protein interaction. Initially, the length of the linker in the inhibitory α-MOrPH will be optimized for maximal activity toward disrupting the PGC1β-CBP80 interaction (e.g. using SP1-3, FIG. 4).

### Optimize In Vitro Potency and Cell Permeability of Inhibitory α-MOrPHs

Next, the peptide sequence encompassed by the inhibitory α-MOrPH is optimized to further improve inhibitory potency and cell permeability. Structural insights from the PGC1β-CBP80 complex (FIG. 3) are used to guide the rational design of amino acid substitutions at the level of the interfacial residues and the solvent-exposed residues in order to increase binding affinity for CBP80 and enhance cell permeability, respectively, of these compounds. Structure-activity relationship (SAR) data gathered from sequence variants will provide another means to guide the design of α-MOrPHs with improved inhibitory potency.

The inhibitory activity of these second-generation inhibitory α-MOrPH are determined using the fluorescence polarization-based competition assay described in FIG. 5. To assess cell permeability, fluorescently labeled analogs (e.g. Fluo-PGC-M4, FIG. 4) are prepared, and their cellular uptake in mammalian (i.e. human embryonic kidney HEK293 cells) is measured using flow cytometry and confocal fluorescence microscopy according to well established protocols.

The most promising compounds are further tested for proteolytic stability (i.e. half-life, t_{1/2}) in the presence of proteases and in human blood serum.

### Assess Cellular Activities and Antiproliferative Potencies of Lead Inhibitory α-MOrPHs in Breast Cancer Cells

The ability of the macrocyclic CBP80 inhibitors to disrupt the PGC1β-CBP80 interaction in both ER-negative (SkBr3) and ER-positive (MCF7) breast cancer cells are initially assessed using co-immunoprecipitation analyses of α-MOrPH-treated cells. Consistent with the explication that blocking the PGC1β-CBP80 interaction will decrease the growth of both ER-positive (MCF7) and ER-negative (SkBr3) breast cancer cells, the ability of lead inhibitory α-MOrPHs to reduce the proliferative activity (GI₅₀) and viability (LD₅₀) of MCF7 and SkBr3 cells is evaluated. These features are assessed using cell densitometry and MTT-based assays, respectively. To gain further insight into the mechanism of action of lead inhibitory α-MOrPHs, their effect on the transcription and expression of known PGC1β-regulated genes (e.g. EBAG9, FASN1, ENOL1, GAPDH, HK1) are evaluated using RT-qPCR and Western blot analyses, respectively. These studies provide insights on the cellular effects of lead α-MOrPHs, which is key for assessing these compounds as a novel class of anticancer agents. Further, deep transcriptome sequencing of MCF7 and SkBr3 cells are used to define those human genes that are regulated by PGC1β, estrogen or both. Toward these ends, the estrogen-mediated induction rate for MCF7 cells have been defined, functional PGC1β LNAs have been defined, MCF7 and SkBr3 cells stably expressing MYC-tagged PGC1β that either contains (i.e. full-length protein) or lacks the CBP80-interacting region (amino acids 994-1023) are made. Notably, to date there is no whole-transcriptome analysis of PGC1β-responsive genes. This disclosure is the first to undertake this analysis as well as a whole-transcriptome analysis of cells in which the interaction between PGC1β and CBP80 has been disrupted by replacing cellular PGC1β with the same level of PGC1β that lacks the CBP80-interacting region, to which the whole-transcriptome analysis of cells treated with a lead α-MOrPH are compared.

### Characterize Structurally the α-MOrPH-CBP80 Complex

Work on the structural characterization of the PGC1β-CBP80 interaction has yielded a number of workable conditions for the crystallization of the CBP80-CBP20-cap complex bound to linear PGC1β-derived peptides (see, e.g. FIG. 5B). Using these conditions, crystallization of the CBP80-CBP20-cap complex bound to an inhibitory α-MOrPH is solved. The structure of the α-MOrPH-bound complex is solved using X-ray crystal diffraction and molecular replacement methods. These studies provide detailed information about the structure of the α-MOrPH-CBP80 binding interface, which enables the structure-guided optimization of these inhibitors.

### Example 4: CBP80 Binding to Human PGC1α

In determining the significance and mechanism of action of PGC1α binding to CBP80, it is described herein that what is defined herein as the CBP80-binding motif (CBM) of PGC1α is necessary for PGC1α-mediated gene transcription and critical to a number of cellular processes, including myogenesis (i.e. muscle cell differentiation). The data presented herein additionally found that the CBM is not only required for the PGC1α association with elongating RNA polymerase but for almost all of the RNA-binding activity that is attributable to PGC1α as well as the increased in splicing efficiency of target transcripts. While not wishing to be bound to any particular theory, the changes mediated by the CBM may be through recruitment and/or localization of the Mediator complex to the bodies of target transcript.

Human PGC1α Binds Human CBP80 Directly In Vitro, Via a Region that is Conserved Human with PGC1β

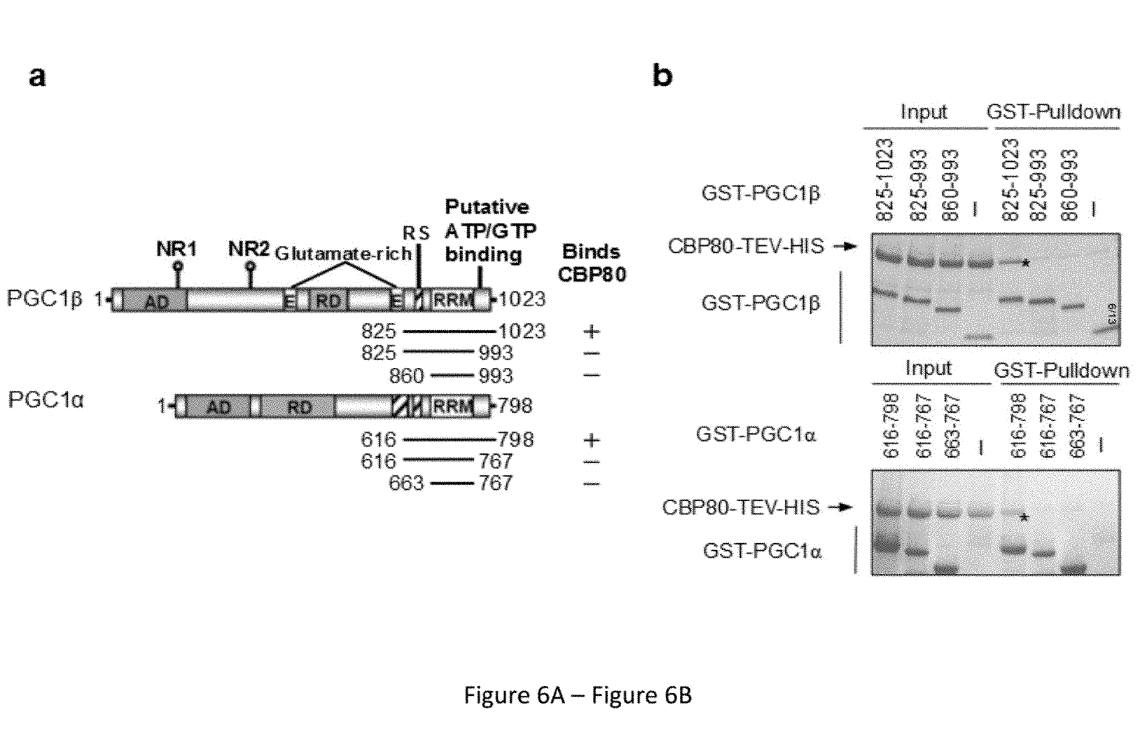

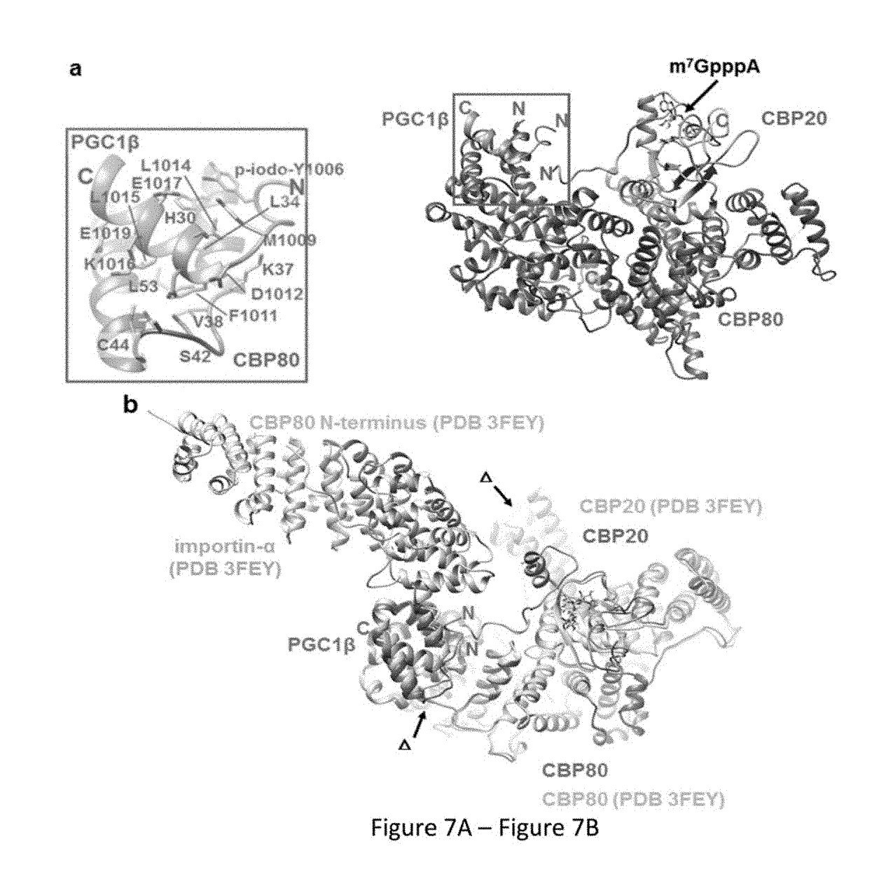

E.coli-produced GST-tagged human PGC1β pulls down baculovirus-produced human CBP80 in vitro, demonstrating a direct interaction between the proteins (See Examples 1-3). To determine whether PGC1α was also able to interact with CBP80 directly in vitro, identical assays were performed using the regions of PGC1α that correspond to the tested regions of PGC1β. As expected based on the similar domain architecture of PGC1α relative to PGC1β, including a perfectly conserved CBM alpha helix, PGC1α binds directly to CBP80 (FIG. 6 and FIG. 7, Table 2).

TABLE 2
Data Collection and Refinement Statistics

| | PGC1β-CBP80-CBP20-m⁷GpppA | |
|---|---|---|
| Data collection | | |
| Space group | P2₁2₁2₁ | |
| Cell dimensions | | |
| a, b, c (Å) | 78.106, 111.897, 124.795 | |
| α, β, γ (°) | 90, 90, 90 | |
| Resolution (Å) | 38.99-2.676 | (2.772-2.676) * |
| R_{merge} | 0.05913 | (0.3856) |
| I/σI | 6.17 | (2.04) |
| Completeness (%) | 98 | (81) |
| Multiplicity | 2.0 | (1.9) |
| CC1/2 | 0.994 | (0.635) |
| CC* | 0.998 | (0.881) |

| | |
|---|---|
| Refinement | |
| Resolution (Å) | 38.99-2.676 |
| No. reflections | 30783 |
| R_{work}/R_{free} | 0.1812/0.2401 |
| No. non-H atoms | 7644 |
| Protein | 7318 |
| Ligand/ion | 52 |
| Water | 274 |
| B-factors | |
| Protein | 50.86 |
| Ligand/ion | 78.98 |
| Water | 39.78 |
| R.m.s. deviations | |
| Bond lengths (Å) | 0.004 |
| Bond angles (°) | 0.55 |

Data were collected from a single crystal.
Values in parentheses are for highest-resolution shell

### Endogenous PGC1α Binds to CBP80 and Target Transcripts in Cells

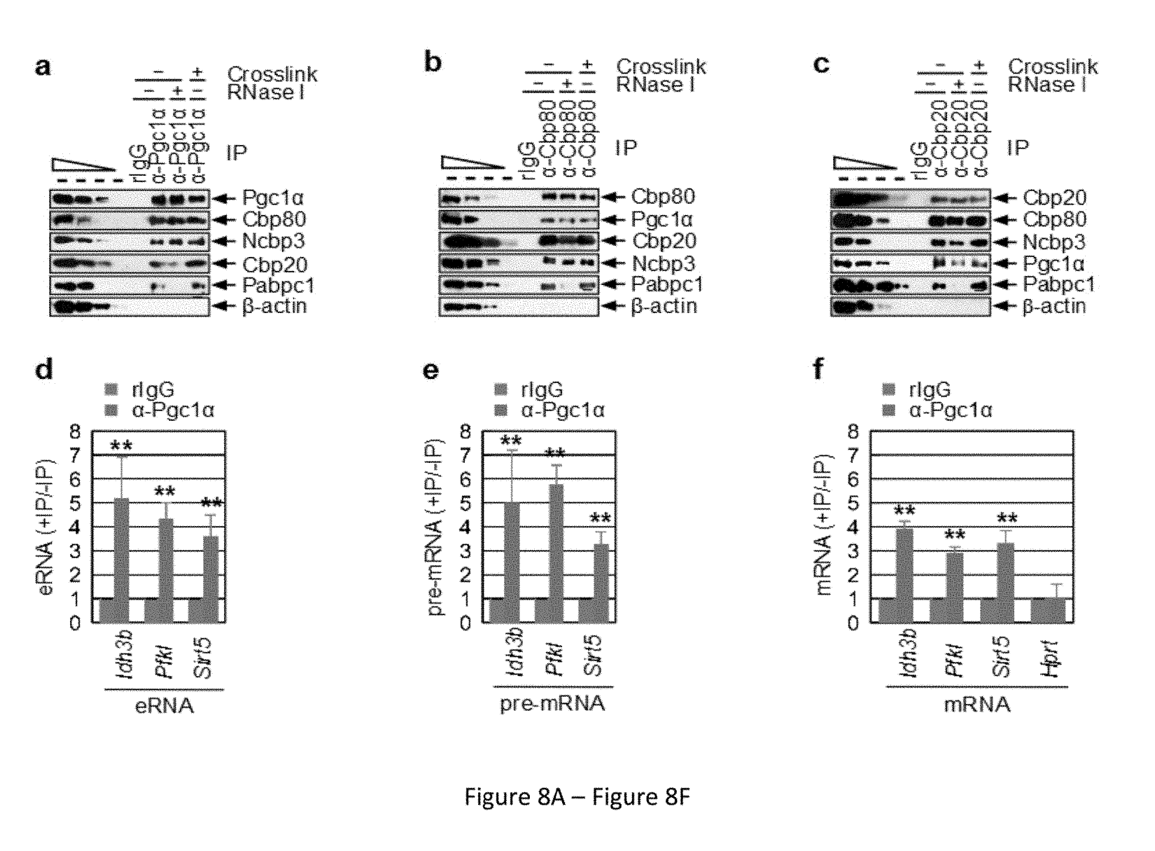

To discern whether the endogenous PGC1α and CBP80 interact in mouse C2Cl2 myoblasts (MBs), for which responsive enhancer RNAs and their corresponding protein-encoding RNAs are known, PGC1α, CBP80 and CBP20 were immunoprecipitated using antibody to each protein. All three proteins co-immunoprecipitate in an interaction that (i) is resistant to RNase I treatment (i.e. persists after cellular RNA is degraded) and (ii) forms in intact cells rather than as an experimental artifact after cell lysis (i.e. in cells crosslinked with formaldehyde prior to lysis) (FIG. 8A-FIG. 8C). Furthermore, using reverse transcriptase coupled to quantitative PCR (RT-pPCR) and primers to known PGC1α-induced target eRNAs, pre-mRNAs and mRNAs purified after immunoprecipitation (IP) of PGC1α, it was determined that PGC1α indeed binds predicted eRNA, pre-mRNA and mRNA target transcripts (FIG. 8D-FIG. 8F).

The CBM of PGC1α is Required for Association with Elongating RNA Polymerase, Co-Activation, and Target RNA Binding

To determine the significance of what is termed herein the CBP80-binding motif (CBM), which consists of PGC1α amino acids 785-793, as defined using X-ray crystallography (FIG. 7), in the context of target gene transcription, first endogenous PGC1α was stably knocked-down using a lentivirus expressing PGC1α shRNA and then different shRNA-resistant, FLAG-tagged variants of PGC1α were expressed. Using this approach, the effect of deleting specific domains from or introducing different mutations within FLAG-PGC1α can be studied without overexpressing the protein relative to cellular PGC1α.

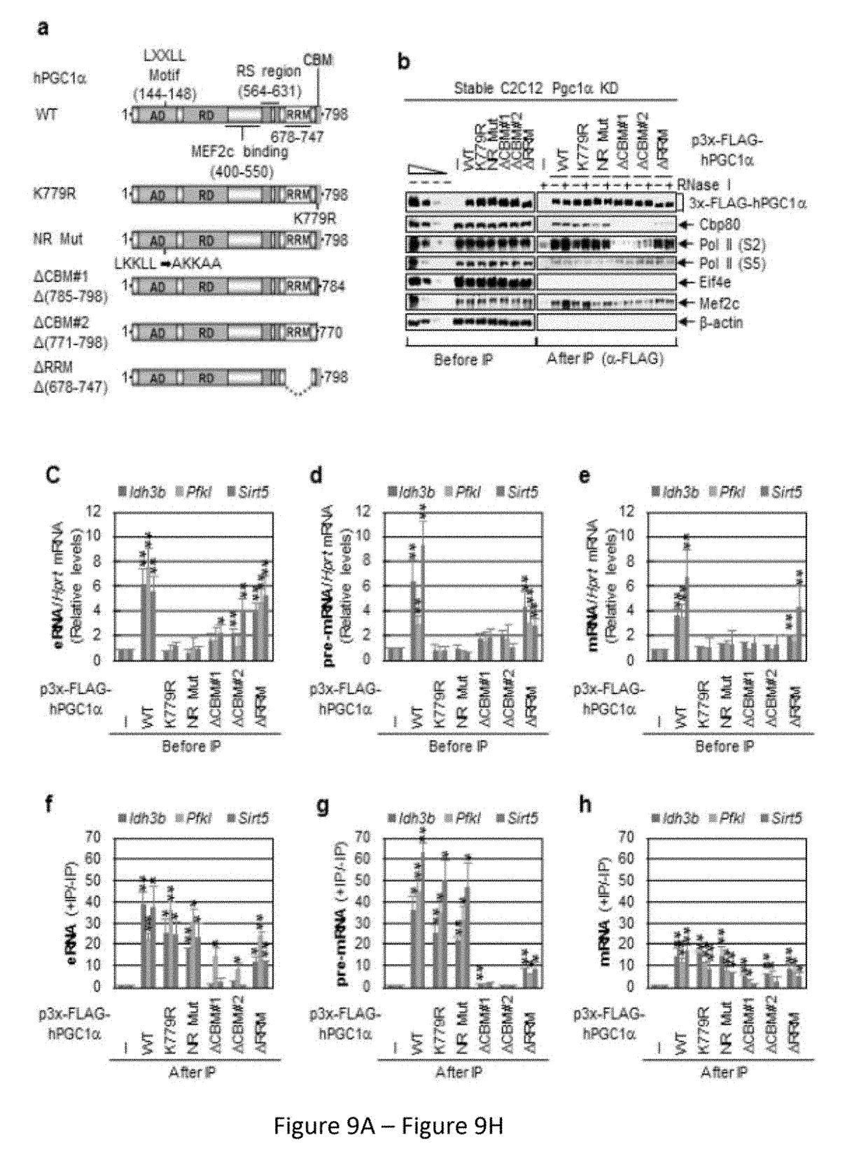

As a negative control for replacement of PGC1α, two variants that abrogate target-gene transcription were expressed: a variant that cannot be methylated at site directly N-terminal to the CBM that was shown to be important for PGC1α-mediated activation of eRNAs (Aguilo et al. 2016), and a variant that cannot bind chromatin remodelers or nuclear receptors (Kressler et al. 2002). These two variants are termed K779R (unable to be methylated) and NR mut (unable to bind nuclear receptors), respectively (FIG. 9A). In addition to these two controls, two CBM deletions were generated: one deletion encompasses only the alpha helix (ΔCBM#1) (FIG. 7), and the other deletes the alpha helix together with the region N-terminal to the alpha helix that contains the K779 methylation site (ΔCBM#2) (FIG. 9A). A deletion of the RNA-recognition motif (ΔRRM) was also generated (FIG. 9A) because of its potential to serve as an RNA-binding domain.

Western blotting of IPs of lysates of C1C12 KD MBs expressing each of these variants revealed that the CBM is responsible for the interaction with CBP80 and that the RRM deletion decreases the co-IP of FLAG-PGC1α with CBP80 to a lesser extent (FIG. 9B). While not wishing to be bound to any particular theory, the RRM may bind directly downstream of the cap or that deletion of the RRM disrupts the structure of the CBM. It was also found that every variant containing the CBM co-immunoprecipitates with elongating RNA polymerase, as detected by blotting for an important phosphorylation site on RNA polymerase II (FIG. 9B; Pol II (S2)).

Using RT-qPCR to detect changes in target gene expression, it was found that variants lacking the CBM, as well as the two negative controls, fail to induce expression of target gene transcripts (FIG. 9C-FIG. 9E). It was additionally found that although there is no increase in gene expression associated with each negative control, they are still able to bind target RNAs as long as the CBM is intact (FIG. 9F-FIG. 9H). The variant lacking the RRM was still able to bind RNA. Taken together, these results show that the CBM of PGC1α is required to induce target gene expression as well as to bind target eRNAs, pre-mRNAs and mRNAs.

PGC1α Binds RNAs Exclusively at their Cap or 5′ End

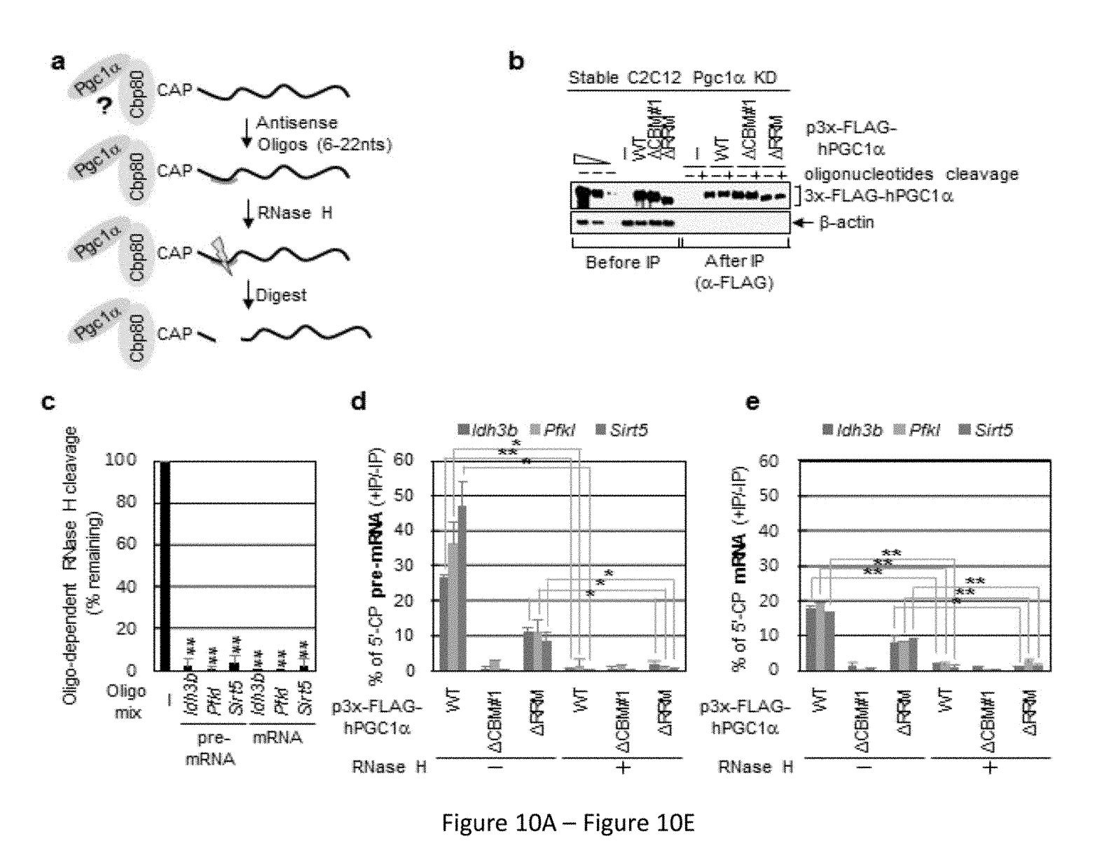

Since the RRM influences target RNA binding, and since there are other domains in PGC1α that are presumed to interact with other RNA processing factors, it was necessary to assess whether the CBM was the primary mode of target RNA binding, i.e. binding occurred primarily via cap-bound CBP80. Thus, DNA oligonucleotides were used to direct RNase H-mediated cleavage of target pre-mRNAs immediately downstream of their cap (FIG. 10A) after IP of wild type (WT), ΔCBM, or ΔRRM variants of FLAG-PGC1α (FIG. 10B). It was found that after cleavage directly downstream of the cap, no variant of PGC1α still appreciably bound RNA, indicating that CBP80 binding is the primary mechanism by which PGC1α binds target RNAs (FIG. 10C-FIG. 10E).

### The CBM of PGC1α is Required for Efficient Myogenesis

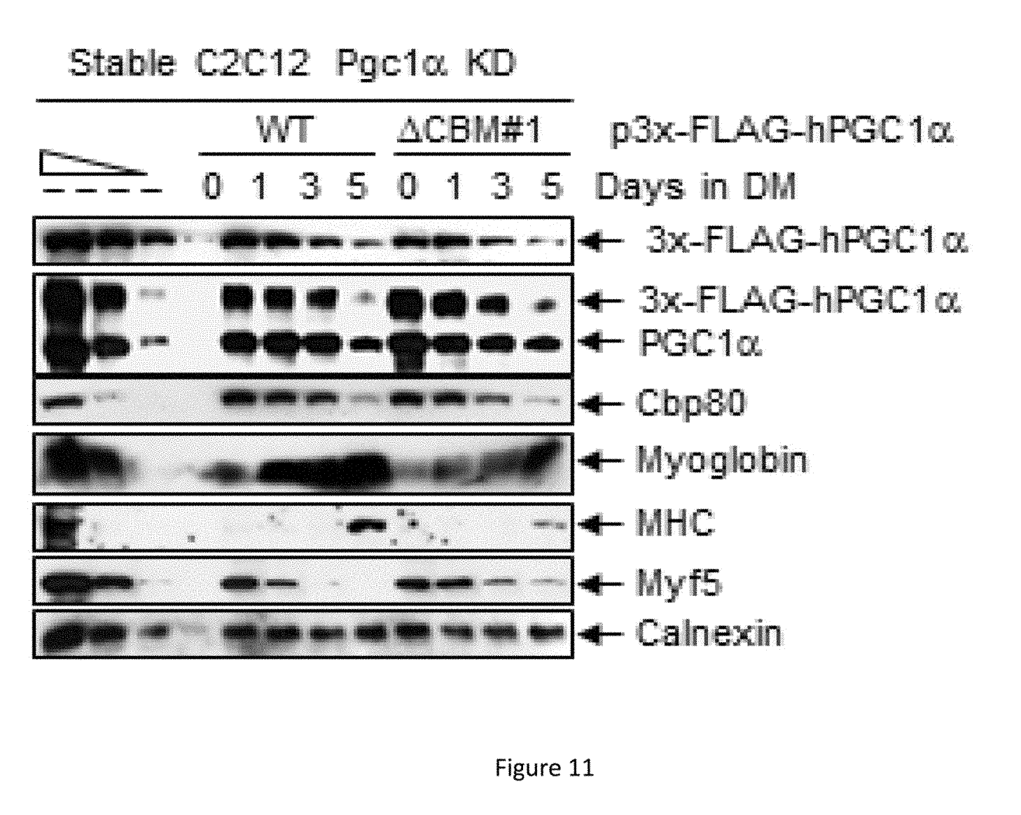

Transcriptional programming and metabolic adaptation is necessary for many different cellular processes, and PGC1α is known to be involved in the transcriptional induction of metabolic genes. It follows that the CBM of PGC1α would influence these cellular processes such as the differentiation of MBs, i.e. muscle-cell precursors, into myotubes (MTs), i.e. mature muscle cells. To test this hypothesis, the C2C12 PGC1α KD MBs expressing either FLAG-PGC1α WT or FLAG-PGC1α ΔCBM were cultured in low levels of horse serum to induce myogenesis and assayed for the loss of MB marker protein expression and the gain of MT protein expression at 1, 3, and 5 days thereafter. As evidence that the CBM of PGC1α is critical for efficient myogenesis, relative to FLAG-PGC1α expression, FLAG-PGC1α ΔCBM expression slowed myogenesis: FLAG-PGC1α ΔCBM expression delayed the production of myoglobin and myosin heavy chain (MHC) proteins, each of which is upregulated during the process of differentiation, and reduced the rate at which the MB marker protein, Myf5, disappeared (FIG. 11).

### Example 5: Peptide Inhibitors of CBP80

Because of the involvement of the PRC1 family coactivators in various human pathologies, it is contemplated herein that agents capable of disrupting the interaction between members of the PRC1 family of coactivators and CBP80 could provide therapeutic benefits. For example, PGC1β binds the estrogen receptor (ER) and promotes the growth of ER-positive breast cancers. PGC1β is also known to rev up the metabolism of ER-negative breast cancer cells by binding the ER-related receptor (ERR). Accordingly, compounds that are capable of disrupting the interaction between PGC1β and CBP80 such as the PGC1β-derived peptide of this invention are expected to interfere with PGC1β-mediated transcriptional function in breast cancer cells, thereby affecting the viability of these cells. Blockage of the interaction of PGC1β with CBP80 is expected to result in an inhibition of the expression of genes that are responsive to ER and ERR in ER-positive breast cancer cells and those responsive to ERR in ER-negative breast cancer cells. This mechanism of action would differ from that of therapeutic agents currently in use for treating ER-positive breast cancer and would provide an entirely new strategy for targeting ER-negative breast cancer.

As described in Example 2, X-ray crystallographic analysis of the complex between CBP80 and the C-terminal domain of PGC1β has revealed that the latter interacts with CBP80 by adopting a short, α-helical structure. Based on this structural information, the inventors have hypothesized that linear and macrocyclic peptides encompassing the CBP80-binding domain of PGC1β (herein referred to also as ‘PGC1β mimics’) could provide viable agents to disrupt the interaction between CBP80 and PGC1β in the context of therapeutic applications. Since the CBP80-binding region in PGC1β shares high sequence homology with corresponding regions in other members of the PGC1 family of transcriptional co-activators (e.g., PGC1α, PRC), it can be derived that the PGC1β-binding domain in CBP80 is also involved in the interaction with other members of the PGC1 family. Accordingly, linear or macrocyclic PGC1β mimics are expected to target and prevent the interaction of CBP80 with any member of the PGC1 transcriptional co-activator family.

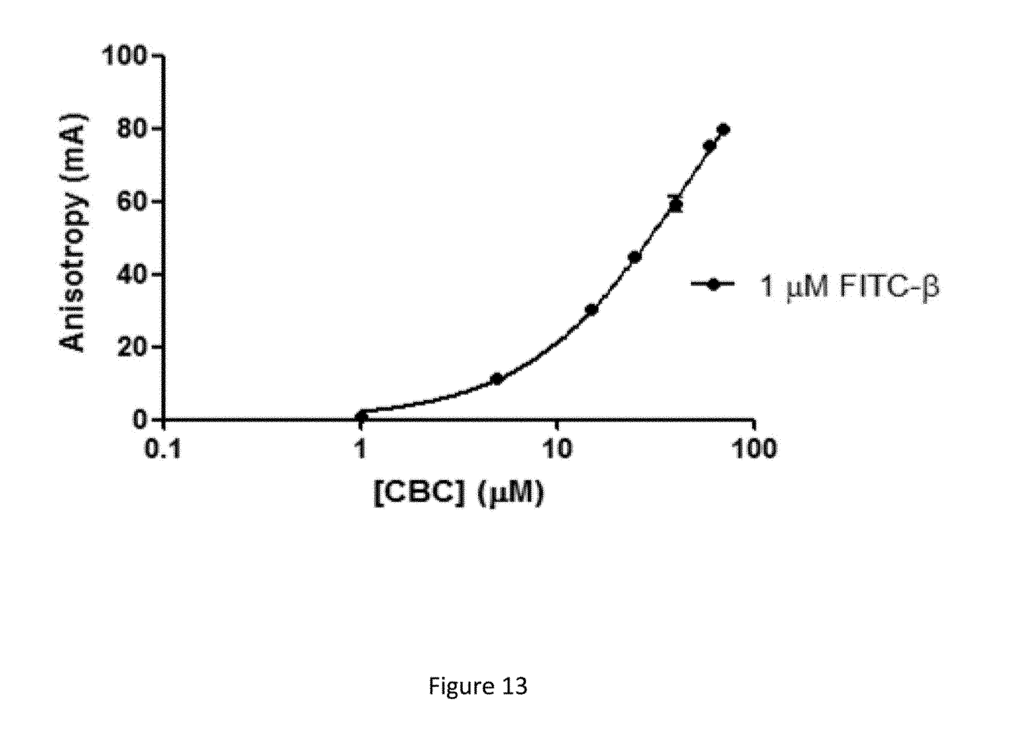

In order to develop a first set of PGC1β mimics, a series of linear peptides encompassing the CBP80-binding domain of PGC1β were designed and synthesized (Table 3). The ability of these peptides to bind to CBP80 in complex with CBP20 (also referred herein as ‘CBC complex’ or simply ‘CBC’) was assessed using a fluorescence polarization assay in the presence of fluorescein-conjugated PGC1β-derived peptide (FITC-Ahx-AMDFDSLLKEAQQSLH-NH₂ (SEQ ID NO:XX), where FITC is fluorescein-isothiocyanate and Ahx is 6-aminohexanoic acid). Titration of CBC complex in the presence of the fluorescein-conjugated PGC1β-derived peptide resulted in an increase in fluorescence polarization (FP) that derives from a reduction in the mobility of the fluorescein fluorophore as the peptide binds to protein. From a plot of the fluorescence polarization signal measured at varying concentrations of CBC complex, an equilibrium dissociation constant (K_{D}) of 47 vμM was determined for interaction of CBP80 with the PGC1β-derived peptide (FIG. 13).

The relative activity of the PGC1β mimics toward disrupting the interaction between CBC and the fluorescein-conjugated PGC1β-derived peptide was measured in a competition assay. Briefly, a fixed amount of CBC and fluorescein-conjugated PGC1β-derived peptide are incubated with increasing amounts of the PGC1β mimic (FIG. 11A). From the resulting dose-dependent inhibition curves, the in vitro CBC inhibitory activity of the PGC1β mimic compound is measured based on their respective half-maximal inhibitory concentration (IC₅₀) (FIG. 5B). Using this assay, various PGC1β mimics were evaluated for their relative ability to target CBC and disrupt its binding interaction with PGC1β. As shown by the data in Table 3, both an unlabeled version of the PGC1β peptide (PGC1β-Cterm peptide) and a biotinylated version thereof (Biot-PGC1β peptide) were found to inhibit CBC with an IC₅₀ of about 35 μM. Short peptides derived from the C-terminal domain of PGC1β and PRC proteins, two other members of the PGC1 family of transcriptional coactivators, were also to bind CBC and inhibits its interaction with the fluorescein-labeled PGC1β peptide with IC₅₀ values in the low micromolar range. These results demonstrate that the C-terminal domains of these proteins indeed share an identical binding site in the CBP80 protein. An implication of these results is that PGC1 mimics can disrupt the interaction of CBP80 with all known members of the PGC1 family of transcriptional coactivators.

A shorter (11mer) version of the PGC1β-derived peptide called ‘PGC1β-wt’ was found to retain significant inhibitory activity against CBP80 as part of the CBC complex (IC₅₀: 69 μM, Table 3). To examine the tolerance of this compound to sequence modification, an alanine-scanning library was synthesized. This library of PGC1β mimics consisted of 11 linear peptides (PGC1β-2A through PGC1β-12A; Table 3), in which each amino acid residue of the parent PGC1β-wt peptide was replaced with alanine (or with glycine, when the parent residue is alanine). The peptides were tested for their ability to inhibit the CBC-PGC1β interaction using the aforementioned fluorescence polarization-based competition assay. As summarized in Table 3, these experiments showed that residues Phe3, Asp4, Leu6, Leu7, and Ala10 are energetically important for the interaction with CBP80 in the CBC complex, as evinced from the significant drop in inhibitory activity (IC₅₀>200 μM) upon substitution of each of these positions with alanine (or glycine for Ala10) when compared to the reference peptide PGC1β-wt. In contrast, alanine replacement of Ser5, Lys8, and Gln11 had no effect on the inhibitory activity of the PGC1β-wt peptide, indicating that these sites can tolerate substitution with both canonical or non-canonical amino acid residues. Interestingly, alanine replacement of Asp2, Glu10, and Gln12 improved the inhibitory activity of the PGC1β mimic up to 2-fold as compared to the reference peptide PGC1β-wt. Further PGC1β mimics containing multiple sequence substitutions (PGC1β-13 through PGC1β-18; Table 3) were found to block the CBC-PGC1β interaction with micromolar activity. These compounds include PGC1β mimics containing non-canonical amino acids such as 2-aminoisobutyric acid (Aib) and N7-crotonyl-2,4-diaminobutyric acid (CrtDab). Aib is an α,α-disubstituted amino acid which is well known for its ability to stabilize α-helical peptide structures as well as increase peptide stability against proteolysis.

CrtDab is a non-canonical amino acid containing a side-chain electrophilic group (i.e., crotonyl group). Based on the structure of the CBC-PGC1β peptide complex (FIG. 3), the side-chain amino group of Lys8 in the PGC1β peptide is expected to lie in close proximity (<3-4 Å) to a cysteine residue in the CBP80 protein (i.e., Cys44). Accordingly, PGC1β mimics containing an electrophilic side-chain group at the position corresponding to Lys8 (e.g., PGC1β-18 in Table 3) can potentially form a covalent complex with the CBP80 protein (i.e., via Michael addition of Cys44 thiol group to the crotonyl group of CrtDab residue in PGC1β-18 peptide), which should result in a large potentiation of their CBP80 inhibitory activity.

Altogether, the results presented herein demonstrate that short peptides derived from the C-terminal domain of PGC1β, PGC1α, and PRC are viable inhibitors of the CBP80 protein in the CBC complex. Furthermore, with respect to the design of linear or macrocyclic PGC1β mimics based on these sequences, the structure-activity data gathered from the alanine-scanning experiments indicate that (a) the positions corresponding to Phe3, Asp4, Leu6, Leu7, and Ala10 in the PGC1β-wt peptide are expected to tolerate substitutions with canonical or non-canonical amino acid residues that are identical or closely related in structure to the parental amino acid residues found in the PGC1β-, PGC1α-, or PRC-based peptide; (b) the positions corresponding to Asp2, Ser5, Lys8, Glu10, Gln11, and Gln12 are tolerant to substitutions with canonical or non-canonical amino acid residues that are both similar and different in structure from the parental amino acid residues found in the PGC1β-, PGC1α-, or PRC-based peptide; and (c) the positions corresponding to Asp2, Glu10, and Gln12 could benefit from substitutions with alanine and other canonical or non-canonical amino acid residues that stabilize α-helical peptide structures (e.g., Aib).

TABLE 3
Sequence and in vitro inhibitory
activity of linear PGC1β mimics.

| | | IC₅₀ |
|---|---|---|
| Peptide | Sequence^{a} | (μM)^{b} |
| Biot-PGC1β | Biot-Ahx-SGG-AMDFDSLLKEAQ | 35 ± 2 |
| | QSLH-NH₂ (SEQ ID NO: 26) | |
| PGC1β-Cterm | Ac-AMDFDSLLKEAQQSLH-NH₂ | 32 ± 3 |
| | (SEQ ID NO: 27) | |
| PGC1α-Cterm | Ac-SLDFDSLLKEAQRSLRR-NH₂ | 26 ± 3 |
| | (SEQ ID NO: 28) | |
| PRC-Cterm | Ac-SLDFDDLLKQAQKNLRR-NH₂ | 93 ± 11 |
| | (SEQ ID NO: 29) | |
| PGC1β-wt | Ac-MDFDSLLKEAQQS-NH₂ | 69 ± 3 |
| | (SEQ ID NO: 30) | |
| PGC1β-2A | Ac-MAFDSLLKEAQQS-NH₂ | 50 ± 1 |
| | (SEQ ID NO: 31) | |
| PGC1β-3A | Ac-MDADSLLKEAQQS-NH₂ | >200 |
| | (SEQ ID NO: 32) | |
| PGC1β-4A | Ac-MDFASLLKEAQQS-NH₂ | >200 |
| | (SEQ ID NO: 33) | |
| PGC1β-5A | Ac-MDFDALLKEAQQS-NH₂ | 64 ± 2 |
| | (SEQ ID NO: 34) | |
| PGC1β-6A | Ac-MDFDSALKEAQQS-NH₂ | >200 |
| | (SEQ ID NO: 35) | |
| PGC1β-7A | Ac-MDFDSLAKEAQQS-NH₂ | >200 |
| | (SEQ ID NO: 36) | |
| PGC1β-8A | Ac-MDFDSLLAEAQQS-NH₂ | 72 ± 4 |
| | (SEQ ID NO: 37) | |
| PGC1β-9A | Ac-MDFDSLLKAAQQS-NH₂ | 33 ± 2 |
| | (SEQ ID NO: 38) | |
| PGC1β-10A | Ac-MDFDSLLKEGQQS-NH₂ | >200 |
| | (SEQ ID NO: 39) | |
| PGC1β-11A | Ac-MDFDSLLKEAAQS-NH₂ | 64 ± 2 |
| | (SEQ ID NO: 40) | |
| PGC1β-12A | Ac-MDFDSLLKEAQAS-NH₂ | 56 ± 1 |
| | (SEQ ID NO: 41) | |
| PGC1β-13 | Ac-MAFDSLLKNAAAS-NH₂ | <100 |
| | (SEQ ID NO: 42) | |
| PGC1β-14 | Ac-MAFDSLLKNAAQS-NH₂ | <100 |
| | (SEQ ID NO: 43) | |
| PGC1β-15 | Ac-MAFDSLLKAAQQS-NH₂ | <100 |
| | (SEQ ID NO: 35) | |
| PGC1β-16 | Ac-MAFDSLLK(Aib)AQQS-NH₂ | <100 |
| | (SEQ ID NO: 36) | |
| PGC1β-17 | Ac-MAFDSMLKAAQQS-NH₂ | <100 |
| | (SEQ ID NO: 37) | |
| PGC1β-18 | Ac-MDFDSLL(CrtDab)EAQQ | <100 |
| | S-NH₂ (SEQ ID NO: 37) | |
| PGC1β-19 | Ac-MDFDSLLKEAQQSRRRRRR | 80 ± 10 |
| | RR-NH₂ (SEQ ID NO: 38) | |

^{a}Ac = acetyl; Biot = biotin; Ahx = 6-aminohexanoic acid; Aib = 2-aminoisobutyric acid; CrtDab = N^{γ}-crotonyl-2,4-diaminobutyric acid. C-terminal ‘-NH₂’ refers to amidated C-terminal end of the peptide.
^{b}IC₅₀ was determined using fluorescence polarization based competition assay in the presence of 1 μM of FITC-labeled PGC1β (FITC-Ahx-AMDFDSLLKEAQQSLH-NH₂, where FITC is fluorescein-isothiocyanate) and 30 μM of the CBP80/CBP20 complex in HEPES buffer (10 mM HEPES, 50 mM NaCl, 5 mM BME, pH 7.5).

The materials and methods are now described.

Peptide Synthesis

The compounds Biot-PGC1β, PGC1β-Cterm, PGC1α-Cterm, and PRC-Cterm were obtained via custom-made synthesis from SynPeptides. The other PGC1β mimics described in Table 3 were synthesized on a Rink Amide resin (0.7 mmol/g; Novagen) using the standard Fmoc-based solid phase peptide synthesis at a 25 μmol scale. Resins were deprotected by two 15-min incubations with 30% piperidine in dimethyformamide (DMF). The C-terminal amino acid was manually coupled to the deprotected resin using Fmoc-protected amino acids (5 equivalents), 1-(cyano-2-ethoxy-2-oxoethylidenaminooxy) dimethylamino-morpholino-carbenium hexafluorophosphate (COMU; 4.97 equivalents), and N,N-diisopropylethylamine (DIPEA; 10 equivalents) in DMF for one hour. The peptides were then synthesized using an automated microwave-assisted peptide synthesizer (Liberty, CEM) and acetylated at the N-terminus by incubation with 20% acetic anhydride in DMF for 1 hour. The final deprotection and cleavage of peptides were performed in Deprotection Solution (3 ml of 95% (v/v) trifluoroacetic acid, 2.5% triisoproplysilane, and 2.5% water) with stirring at room temperature for 1.5 hours. The peptides were precipitated in 10 volumes of chilled diethyl ether and purified using a C₁₈ Reverse-Phase HPLC (Agilent Technologies). The purity and identity of the peptides were confirmed by analytical RP C₁₈-HPLC analysis and MALDI-TOF mass spectrometry (Bruker Autoflex II), respectively.

TABLE 4
MS data and HPLC elution times for the linear PGC1β mimics.

| | | |
|---|---|---|
| m/z | HPLC | Ret. Time |

| Peptide | Calc. | Exp. | method^{a} | (min) |
|---|---|---|---|---|
| PGC1β-wt | 1552.7 | 1552.3 | 1 | 21.4 |
| PGC1β-2A | 1508.7 | 1508.3 | 1 | 21.4 |
| PGC1β-3A | 1476.7 | 1476.3 | 1 | 19.3 |
| PGC1β-4A | 1508.7 | 1510.1 | 1 | 22.1 |
| PGC1β-5A | 1536.7 | 1538.5 | 1 | 21.7 |
| PGC1β-6A | 1510.7 | 1510.5 | 1 | 19.2 |
| PGC1β-7A | 1510.7 | 1510.6 | 1 | 19.4 |
| PGC1β-8A | 1495.6 | 1495.5 | 1 | 22.0 |
| PGC1β-9A | 1494.7 | 1494.7 | 1 | 21.7 |
| PGC1β-10A | 1538.7 | 1538.7 | 1 | 20.1 |
| PGC1β-11A | 1495.7 | 1495.7 | 1 | 22.0 |
| PGC1β-12A | 1495.7 | 1495.7 | 1 | 21.5 |
| PGC1β-13 | 1379.7 | 1380.0 | 2 | 18.8 |
| PGC1β-14 | 1436.7 | 1437.1 | 2 | 18.3 |
| PGC1β-15 | 1450.7 | 1452.0 | 2 | 23.4 |
| PGC1β-16 | 1464.7 | 1465.4 | 2 | 20.7 |
| PGC1β-17 | 1467.7 | 1469.0 | 2 | 18.2 |
| PGC1β-18 | 1592.7 | 1594.7 | 2 | 23.1 |

^{a}Method 1: VisionHT C18 RP column (Grace; 5 μM, 250 mm × 4.6 mm); 1 ml/min; 0 min: 5% buffer B, 3 min: 5% buffer B, 20 min: 75% buffer B, 22 min: 95% buffer B. Buffer A: water + 0.1% TFA, Buffer B: acetonitrile + 0.1% TFA. Method 2: Column: Vydac C18 RP column (Grace; 5 μM, 250 mm × 4.6 mm); 1 ml/min; 0 min: 5% buffer B, 5 min: 20% buffer B, 25 min: 50% buffer B, 30 min: 95% buffer B. Buffer A: water + 0.1% TFA, Buffer B: acetonitrile + 0.1% TFA.

Fluorescence Polarization Assay

The assay was performed in a 15-μl scale in a low-volume 384-well black plate on a Tecan M1000 Plate Reader using 470/20 nm excitation and 520/20 nm emission filters. All proteins and peptides were dissolved and diluted in HEPES buffer (50 mM NaCl, 10 mM HEPES [pH 7.5, 5] mM, and 2-mercaptoethanol). Fluorescently labelled PGC1β peptide (FITC-Ahx-AMDFDSLLKEAQQSLH-NH₂) was used at 1 μM final concentration, and fluorescence polarization data were recorded at increasing concentration of the CBC complex (1-80 μM) after a 10-min incubation. The resulting concentration-dependent FP curve was fit to a 1:1 binding model to yield dissociation constant (K_{D}) values. Measurements were performed at least in in duplicates.

IC₅₀ Determinations

IC₅₀ values were determined by adapting the fluorescence polarization assay described above to a competition format. 1 μM of the FITC-labeled PGC1β peptide (FITC-Ahx-AMDFDSLLKEAQQSLH-NH₂) was incubated with 30 μM of the CBP80-CBP20 complex in HEPES Buffer (10 mM HEPES [pH 7.5], 50 mM NaCl and 5 mM 2-mercaptoethanol). The inhibitory peptide was added to the solution at varying concentrations, typically ranging from 1 to 200 μM. Half-maximal inhibitory concentration (IC₅₀) values were calculated from the resulting inhibition curves as given by dose-dependent decrease in the fluorescence polarization signal. All measurements were performed at least in in duplicates.

### Example 5: Cyclic Peptide Inhibitors of CBP80

Representative cyclic peptide inhibitors of CBP80 were prepared via cyclization of a PGC1β-derived peptide (Ac-CAFDSLLKCAQQS-NH₂) via cysteine alkylation with various cross-linking agents (Table 5). These compounds were designed based on structural insights as derived from inspection of the crystal structure of PGC1β C-terminal domain bound to CBP80 (FIG. 2). In particular, these analyses suggested that cyclic PGC1β mimics could be generated based on the CBP80-binding domain of PGC1β by replacing two solvent-exposed residues (corresponding to Met1009 and Glu1017 in PGC1β) with cysteines followed by inter-side-chain cross-linking with a cysteine-alkylating agent. As described in Table 5, cross-linking agents deemed suitable for this purpose include 1,4-bis(bromomethyl)benzene, 1,3-bis(bromomethyl)benzene, 1,2-bis(bromomethyl)benzene, 1,4-dibromo-but-2-ene, and 1,4-dibromo-butane. In the corresponding cyclic PGC1β mimics, the inter-side-chain linkage spans a distance that approximately matches the distance between the α-carbons of the corresponding residues in the CBP80-bound PGC1β-derived peptide. Similarly, other cyclization strategies can be applied to generate cyclic PGC1β mimics for the purpose of inhibiting CBP80. For example, cyclic PGC1β mimics featuring an inter-side-chain i/i+4 linkage can be generated by replacing two suitable (i.e., solvent-exposed) residues in a PGC1β-derived peptide (e.g., corresponding to Ser1013/Glu1017 or to Asp1012/Lys016 in PGC1β) with judiciously chosen amino acids capable of forming a i/i+7 inter-side-chain linkage via a lactam (e.g., Lys/Glu, Lys/Asp, Glu/Lys, or Asp/Lys), triazole, or hydrocarbon staple (e.g., 2-(2-propenyl)alanine/2-(4-pentenyl)alanine, allyl-glycine/2-(4′-pentenyl)glycine), 2-(4-pentenyl)alanine/2-(2-propenyl)alanine, or 2-(3-butenyl)alanine/2-(3-butenyl)alanine). In the corresponding cyclic PGC1β mimics, the inter-side-chain linkage is chosen so that it spans a distance that approximately matches the distance between the α-carbons of the corresponding i and i+4 residues in the CBP80-bound PGC1β-derived peptide. As another example, cyclic PGC1β mimics featuring an inter-side-chain i/i+7 linkage can be generated by replacing two suitable (i.e., solvent-exposed) residues in a PGC1β-derived peptide (e.g., corresponding to Ser1013/Gln1020 in PGC1β) with judiciously chosen amino acids capable of forming a i/i+7 inter-side-chain linkage via a hydrocarbon staple (e.g., (R)-2-(4-pentenyl)alanine/(S)-2-(7-octenyl)alanine, (S)-2-(7-octenyl)alanine/(S)-2-(4-pentenyl)alanine, or (S)-2-(5-hexenyl)alanine/(S)-2-(6-heptenyl)alanine). In the corresponding cyclic PGC1β mimics, the inter-side-chain linkage is chosen so that it spans a distance that approximately matches the distance between the α-carbons of the corresponding i and i+7 residues in the CBP80-bound PGC1β-derived peptide.

Testing of the cyclic PGC1β mimics with SEQ ID NOS:26 through 29 in the competition assay (FIG. 5A) showed that these compounds are able to inhibit CBP80 binding to the fluorescein-conjugated PGC1β peptide with low to mid-low micromolar activity. These results thus demonstrate that cyclic peptides prepared according to the methods disclosed in this invention can be effectively applied to target CBP80 and disrupt its interaction with members of the PGC1 family of transcriptional coactivators.

The materials and methods are now described.

Synthesis of Cyclic PGC1β Mimics

A precursor linear peptide corresponding to Ac-CAFDSLLKCAQQS-NH₂ was synthesized via standard Fmoc-based solid-phase peptide synthesis using a CEM Liberty microwave peptide synthesizer. The peptide was synthesized on a Rink Amide resin (0.7 mmol/g; Novagen) at a 25 μmol scale. The final Fmoc protected peptidyl-resin was deprotected using a solution 30% of Piperidine in DMF, 2 times for 8 minutes. Before the cleavage from the resin, the peptide was N-terminally acetylated exposing 0.1 mmol of resin to 5 mL of a solution of 0.5 M acetic anhydride, 0.15 M DIPEA and 0.02 M HOBt in DMF, 2 times for 15 minutes. The peptide was cleaved from the resin using a solution of TFA/H₂O/TIPS (95:2.5:2.5 v/v/v) for 2 hours. The resin was filtered and the crude peptide was precipitated with diethyl ether, dissolved in H₂O/CH₃CN mixture (1:1 v/v), and lyophilized. The crosslinking reactions were performed dissolving 20 mg of the crude peptide in 2 mL of H₂O/THF (9:1). To this solution 1.5 equivalents of tris(2-carboxyethyl)phosphine (TCEP) and 3 equivalents of K₂CO₃ were added and the reaction was stirred for 2 hours to keep the cysteine residues in the reduced form. 3 equivalents of the cysteine crosslinking reagent (e.g., 1,4-bis(bromomethyl)benzene) was dissolved in 200 μL of THF and added dropwise to the peptide solution, followed by the addition of 6 equivalents of triethylamine. The reaction was stirred overnight and lyophilized. The cyclic peptides were purified by semi-preparative RP-HPLC using a semi-preparative Grace C18 RP column and a linear gradient of H₂O (0.1% TFA)/CH₃CN (0.1% TFA) from 20% to 75% of CH₃CN in 25 minutes at flow rate of 2.5 mL/min. The collected fractions containing the cyclic peptide were lyophilized. The purity and identity of the peptides were confirmed by analytical RP Cis-HPLC analysis and MALDI-TOF mass spectrometry (Bruker Autoflex II), respectively.

TABLE 5
Structure, MS data and HPLC elution times for representative cyclic PGC1β mimics.

| | | | | | Ret. |
|---|---|---|---|---|---|
| | | | m/z | | Time |
| Name | Peptide sequence ^{a} | Linker | Calc. | Exp. | (min) ^{b} |
| PGC1β-m1 | Ac-CAFDSLLKCAQQS-NH₂ (SEQ ID NO: 49) | 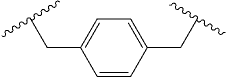 [SMILES: *Cc1ccc(C*)cc1] | 1556.82 | 1557.46 | 12.76 |
| PGC1β-m2 | Ac-CAFDSLLKCAQQS-NH₂ (SEQ ID NO: 49) | 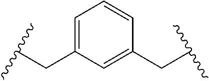 [SMILES: *Cc1cccc(C*)c1] | 1556.82 | 1557.75 | 12.67 |
| PGC1β-m3 | Ac-CAFDSLLKCAQQS-NH₂ (SEQ ID NO: 49) | 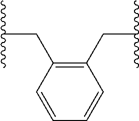 [SMILES: *Cc1ccccc1C*] | 1556.82 | 1557.56 | 12.68 |
| PGC1β-m4 | Ac-CAFDSLLKCAQQS-NH₂ (SEQ ID NO: 49) | 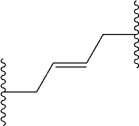 [SMILES: *C/C=C/C*] | 1506.76 | 1507.92 | 11.47 |
| PGC1β-m5 | Ac-CAFDSLLKCAQQS-NH₂ (SEQ ID NO: 49) | 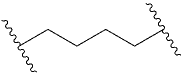 [SMILES: *CCCC*] | 1508.77 | 1509.22 | 11.67 |

^{a} The cysteine residues are crosslinked at the sulfur atom by means of the non-peptidic linker moiety are underlined.
^{b} HPLC analysis were performed using a Grace Vision HT C18 HL column (Grace; 5 μM, 250 mm × 4.6 mm) and a linear gradient of H₂O (0.1% TFA)/CH₃CN (0.1% TFA) from 20% to 70% of CH₃CN (0.1% TFA) in 17 min at a flow rate of 1 ml/min.

### Example 6: Cell Activity Studies

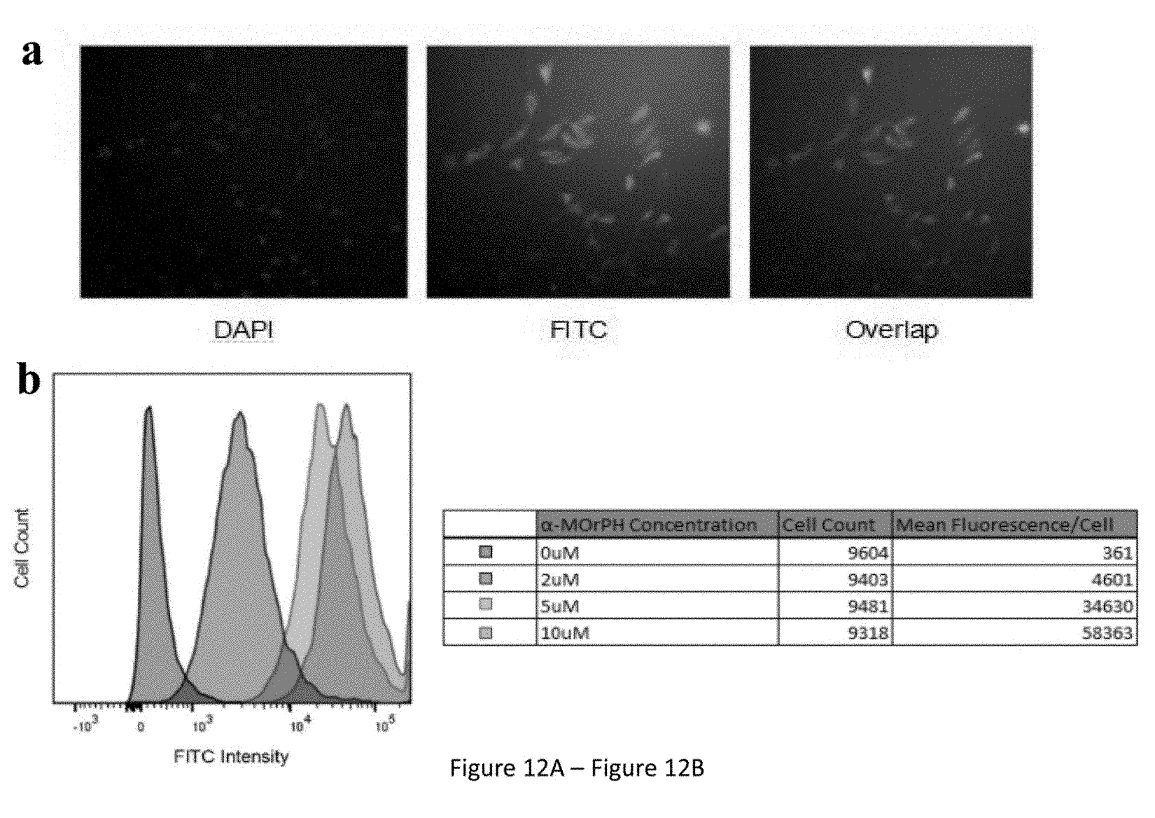

To examine the cellular activity of a representative PGC1β mimics, a fluorescein-conjugated version of the PGC1β-derived peptide corresponding to SEQ ID NO:25 was prepared (FITC-Ahx-MDFDSLLKEAQQSRRRRRRRR-NH₂ (SEQ ID NO:51)). This peptide contains a PGC1β-derived sequence that is C-terminally fused to a cell-penetrating poly-arginine (Arg₈) tag. Cervical cancer cells (HeLa cells) were incubated with varying amounts of the peptide for 30 minutes, followed by chromatin staining with DAPI. DAPI stains nuclei, enabling to quantitate inhibitor fluorescence (from fluorescein fluorophore) relative to nuclear staining. These experiments indicated that PGC1β-derived peptide is able to penetrate cells at low concentration (<5 μM), as determined by flow cytometry (FIG. 12B). In addition, fluorescence from the peptide-linked fluorescein overlapped well with nuclear staining (FIG. 12A), indicating that the PGC1β-derived peptide localized in the nuclei, i.e. in the cellular compartment where CBP80 is found. Similar experiments using a control fluorescein-conjugated polyarginine peptide lacking the PGC1β-derived sequence (i.e., FITC-Ahx-RRRRRRRR-NH₂ (SEQ ID NO:52)) showed good cell penetration but negligible distribution into the nuclei, supporting the ability of the PGC1β-derived peptide to target CBP80 in the nuclear compartment.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A macrocyclic peptide represented by any one of Formula (I)-Formula (V) 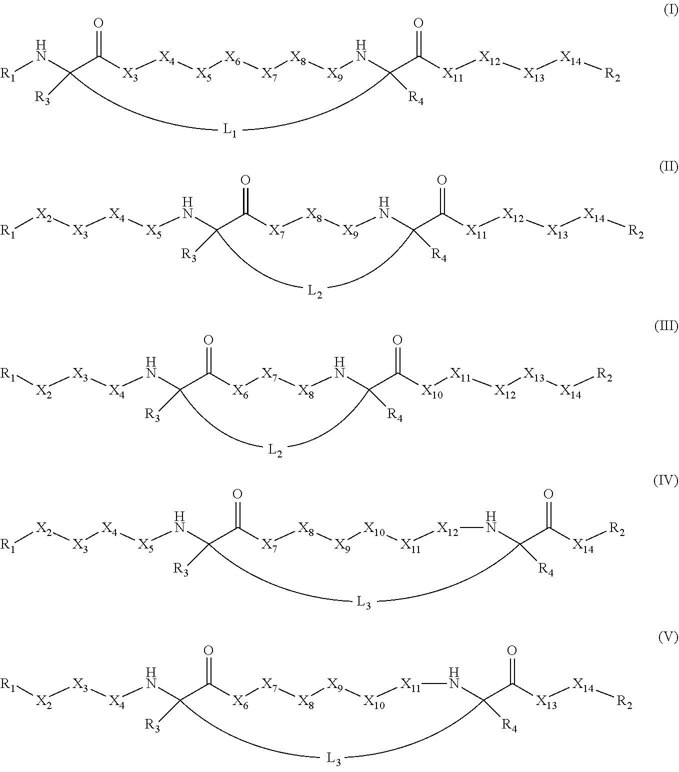
wherein
X₂ is Met, Leu, Ala, Ile, or Val;
X₃ is Asp, Ala, Glu, Asn, Gln, Ser, or Thr;
X₄ is Phe or Ala;
X₅ is Asp, Glu or Ala;
X₆ is Ser, Thr, Ala, Glu, Asp, Gln, Asn, Phe, Tyr, Trp, Met, Leu, Ile, Val, Lys, His, Arg, or Aib;
X₇ is Leu, Ile or Ala;
X₈ is Leu, Ile or Ala;
X₉ is Lys, Ala, Ser, Thr, Glu, Asp, Gln, Asn, Phe, Tyr, Trp, Met, Leu, Ile, Val, His, Arg, Aib, a lysine derivative, a ornithine derivative, or a 2,4-diaminobutyric acid derivative;
X₁₀ is Glu, Gln, Ala, Ser, Thr, Asp, Asn, Phe, Tyr, Trp, Met, Leu, Ile, Val, Lys, His, Arg, or Aib;
X₁₁ is Ala, Gly, Leu, Ile, or Aib;
X₁₂ is Gln, Ala, Asn, Ser, Thr, Glu, Asp, Asn, Phe, Tyr, Trp, Met, Leu, Ile, Val, Lys, His, Arg, or Aib;
X₁₃ is Gln, Arg, Lys, Ala, Ser, Thr, Glu, Asp, Asn, Phe, Tyr, Trp, Met, Leu, Ile, Val, or His;
X₁₄ is Ser, Asn, Ala, Thr, Glu, Asp, Gln, Phe, Tyr, Trp, Met, Leu, Ile, Val, Lys, His, or Arg;
R₁ is hydrogen, an acetyl group, or a label molecule, wherein the label molecule is optionally comprises a spacer unit;
R₂ is a free carboxylic group or an amide group;
R₃ is hydrogen or methyl group;
R₄ is hydrogen or methyl group;
L₁ is a linker unit, such that the linear dimension between the Cα carbon atoms connected by the linker unit is between about 10 and 18 Angstrom units;
L₂ is —(CH₂)₃CH═CH(CH₂)₃— or —(CH₂)₈—; and
L₃ is —(CH₂)₆CH═CH(CH₂)₃— or —(CH₂)₁₁—.

2. The macrocyclic peptide of claim 1, wherein L₁ is selected from the group consisting of:
—CH₂S(CH₂)ₙSCH₂—, wherein n is an integer number comprised between 4 and 8;
—CH₂SCH₂CH═CHCH₂SCH₂—; 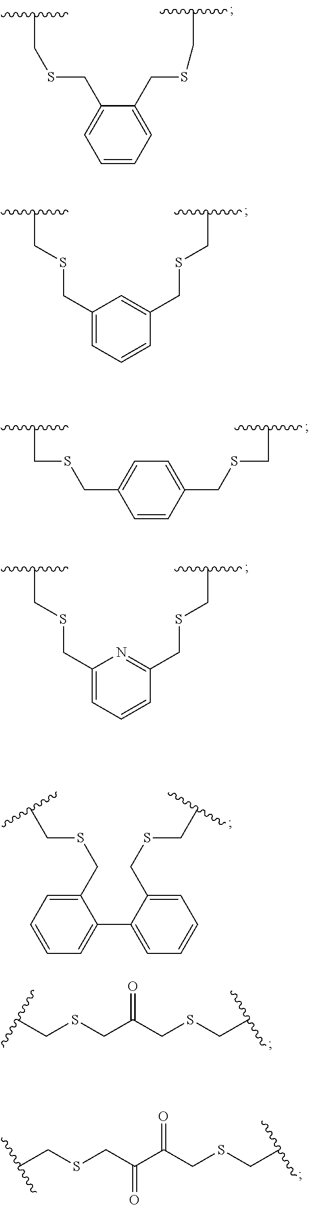
—(CH₂)ₘNHCO(CH₂)ₙ—, wherein m is an integer number between 2 and 4, and n is an integer number between 1 and 2;
—(CH₂)ₙCONH(CH₂)ₘ—, wherein m is an integer number between 2 and 4, and n is an integer number between 1 and 2;
—(CH₂)ₘCH═CH(CH₂)ₙ—, wherein m is an integer number between 1 and 6, and n is an integer number between 1 and 6;
—(CH₂)ₘCH₂CH₂(CH₂)ₙ—, wherein m is an integer number between 1 and 6, and n is an integer number between 1 and 6;
—(CH₂)ₘC≡C(CH₂)ₙ—, wherein m is an integer number between 1 and 6, and n is an integer number between 1 and 6; and
—(CH₂)ₘ(triazole)(CH₂)ₙ—, wherein m is an integer number between 1 and 6, and n is an integer number between 1 and 6.

3. The macrocyclic peptide of claim 1, where in the cyclic peptide is selected from the group consisting of

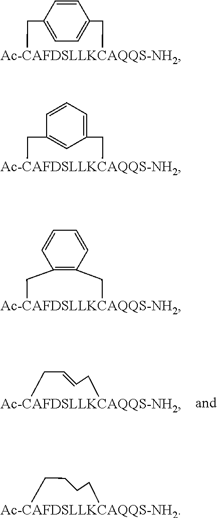

4. The macrocyclic peptide of claim 1, wherein the label molecule is selected from the group consisting of an affinity label molecule, a photoaffinity label, a dye, a chromophore, a fluorescent molecule, a phosphorescent molecule, a chemiluminascent molecule, an energy transfer agent, a photocrosslinker molecule, a redox-active molecule, an isotopic label molecule, a spin label molecule, a metal chelator, a metal-comprising moiety, a heavy atom-comprising-moiety, a radioactive moiety, a contrast agent molecule, a MRI contrast agent, an isotopically labeled molecule, a PET agent, a polypeptide, a cell penetrating polypeptide, a carbohydrate, a polynucleotide, a peptide nucleic acid, a fatty acid, a lipid, biotin, a biotin analogue, a polymer, a small molecule, a drug or drug candidate, a cytotoxic molecule, a solid support, a surface, a resin, a nanoparticle, a quantum dot, and any combination thereof.

5. The macrocyclic peptide of claim 1, wherein the peptide binds human CBP80.

6. The macrocyclic peptide of claim 1, wherein the peptide inhibits the binding of CBP80 to a binding partner.

7. The macrocyclic peptide of claim 6, wherein the binding partner is a member of the PGC1 family of co-activators.

8. The macrocyclic peptide of claim 1, wherein X₉ is selected from the group consisting of a lysine, ornithine and a 2,4-diaminobutyric acid derivative bearing a side-chain electrophilic group capable of reacting with cysteine.

9. The macrocyclic peptide of claim 8, wherein the electrophilic group capable of reacting with cysteine is selected from the group consisting of 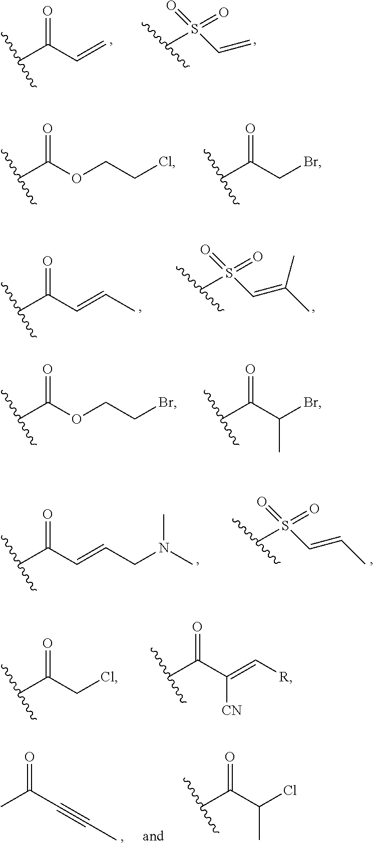
wherein R is selected from the group consisting of a C₃-C₂₀ alkyl, C₃-C₂₀ heteroalkyl, C₆-C₁₀ aryl, and C₆-C₁₀ heteroaryl group.

10. A peptide, wherein the peptide inhibits the binding of CBP80 to a binding partner.

11. The peptide of claim 10, wherein the peptide comprises a sequence selected from the group consisting of X₁X₂X₃X₄X₅X₆X₇X₈X₉X₁₀X₁₁X₁₂X₁₃X₁₄X₁₅X₁₆ (SEQ ID NO: 1) and X₂X₃X₄X₅X₆X₇X₈X₉X₁₀X₁₁X₁₂X₁₃X₁₄ (SEQ ID NO:2),
wherein X₁ is Ala, Ser, or Thr;
X₂ is Met, Leu, Ala, Ile, or Val;
X₃ is Asp, Ala, Glu, Asn, Gln, Ser, or Thr;
X₄ is Phe or Ala;
X₅ is Asp, Glu or Ala;
X₆ is Ser, Thr, Ala, Glu, Asp, Gln, Asn, Phe, Tyr, Trp, Met, Leu, Ile, Val, Lys, His, Arg, or Aib;
X₇ is Leu, Ile or Ala;
X₈ is Leu, Ile or Ala;
X₉ is Lys, Ala, Ser, Thr, Glu, Asp, Gln, Asn, Phe, Tyr, Trp, Met, Leu, Ile, Val, His, Arg, Aib, a lysine derivative, a ornithine derivative, or a 2,4-diaminobutyric acid derivative;
X₁₀ is Glu, Gln, Ala, Ser, Thr, Asp, Asn, Phe, Tyr, Trp, Met, Leu, Ile, Val, Lys, His, Arg, or Aib;
X₁₁ is Ala, Gly, Leu, Ile, or Aib;
X₁₂ is Gln, Ala, Asn, Ser, Thr, Glu, Asp, Asn, Phe, Tyr, Trp, Met, Leu, Ile, Val, Lys, His, Arg, or Aib;
X₁₃ is Gln, Arg, Lys, Ala, Ser, Thr, Glu, Asp, Asn, Phe, Tyr, Trp, Met, Leu, Ile, Val, or His;
X₁₄ is Ser, Asn, Ala, Thr, Glu, Asp, Gln, Phe, Tyr, Trp, Met, Leu, Ile, Val, Lys, His, or Arg;
X₁₅ is Leu; and
X₁₆ is His, or Arg.

12. The peptide of claim 11, wherein the peptide comprises a group on the N-terminus of the peptide, wherein the group is selected from the group consisting of a hydrogen, an acetyl, and a label.

13. The peptide of claim 12, wherein the label is selected from the group consisting of an affinity label molecule, a photoaffinity label, a dye, a chromophore, a fluorescent molecule, a phosphorescent molecule, a chemiluminascent molecule, an energy transfer agent, a photocrosslinker molecule, a redox-active molecule, an isotopic label molecule, a spin label molecule, a metal chelator, a metal-comprising moiety, a heavy atom-comprising-moiety, a radioactive moiety, a contrast agent molecule, a MRI contrast agent, an isotopically labeled molecule, a PET agent, a polypeptide, a cell penetrating polypeptide, a carbohydrate, a polynucleotide, a peptide nucleic acid, a fatty acid, a lipid, biotin, a biotin analogue, a polymer, a small molecule, a drug or drug candidate, a cytotoxic molecule, a solid support, a surface, a resin, a nanoparticle, a quantum dot and any combination thereof.

14. The peptide of claim 10, wherein the peptide comprises a group on the C-terminus of the peptide, wherein the group is selected from the group consisting of a free carboxylic group, an amide group, or a cell penetrating peptide.

15. The peptide of claim 14, wherein the group is a cell penetrating peptide and wherein the cell penetrating peptide is attached to the C-terminus of the peptide optionally via a spacer.

16. The peptide of claim 10, wherein the peptide binds human CBP80.

17. The peptide of claim 10, wherein the binding partner is a member of the PGC1 family of co-activators.

18. The peptide of claim 11, wherein the peptide comprises a sequence selected from the group consisting of AMDFDSLLKEAQQSLH (SEQ ID NO:3), AMDFDSLLKEAQQSLH (SEQ ID NO:4), SLDFDSLLKEAQRSLRR (SEQ ID NO:5), SLDFDDLLKQAQKNLRR (SEQ ID NO:6), MDFDSLLKEAQQS (SEQ ID NO:7), MAFDSLLKEAQQS (SEQ ID NO:8), MDADSLLKEAQQS (SEQ ID NO:9), MDFASLLKEAQQS (SEQ ID NO: 10), MDFDALLKEAQQS (SEQ ID NO: 11), MDFDSALKEAQQS (SEQ ID NO: 12), MDFDSLAKEAQQS (SEQ ID NO: 13), MDFDSLLAEAQQS (SEQ ID NO:14), MDFDSLLKAAQQS (SEQ ID NO:15), MDFDSLLKEGQQS (SEQ ID NO:16), MDFDSLLKEAAQS (SEQ ID NO:17), MDFDSLLKEAQAS (SEQ ID NO:18), MAFDSLLKNAAAS (SEQ ID NO:19), MAFDSLLKNAAQS (SEQ ID NO:20), MAFDSLLKAAQQS (SEQ ID NO:21), MAFDSLLK(Aib)AQQS (SEQ ID NO:22), MAFDSMLKAAQQS (SEQ ID NO:23), MDFDSLL(CrtDab)EAQQS (SEQ ID NO:24), and MDFDSLLKEAQQSRRRRRRRR (SEQ ID NO:25).

19. The peptide of claim 11, wherein X₉ is selected from the group consisting of a lysine, ornithine and a 2,4-diaminobutyric acid derivative bearing a side-chain electrophilic group capable of reacting with cysteine.

20. The peptide of claim 19, wherein the electrophilic group capable of reacting with cysteine is selected from the group consisting of 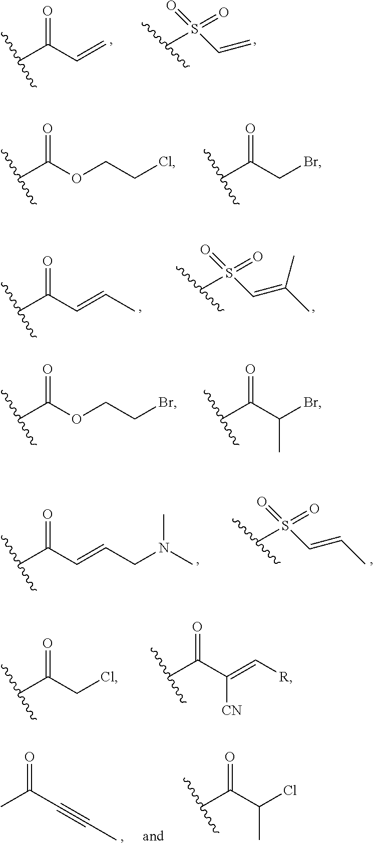
wherein R is selected from the group consisting of a C₃-C₂₀ alkyl, C₃-C₂₀ heteroalkyl, C₆-C₁₀ aryl, and C₆-C₁₀ heteroaryl group.

21. A method for treating or preventing a disease or disorder in a subject in need thereof, the method comprising administering to the subject an effective amount of a composition comprising the peptide of claim 1.

22. The method of claim 21, wherein the disease or disorder is selected from the group consisting of cancer, heart disease, autoimmune disorders, obesity, diabetes, and chronic inflammation disorders.

23. The method of claim 22, wherein the cancer is breast cancer.

24. The method of claim 23, wherein the breast cancer is ER-negative breast cancer.

25. The method of claim 21, wherein the composition further comprises a pharmaceutically acceptable carrier.

26. The method of claim 21, further comprising administering to the subject a second agent.

27. The method of claim 26, wherein the second agent is one selected from the group consisting of immunomodulatory agent, an antineoplastic agent, an anti-angiogenic agent and an anti-diabetic agent.

28. A method for inhibiting the interaction between CBP80 and a PGC1 family of co-activator in a subject in need thereof, the method comprising administering to the subject an effective amount of a composition comprising the peptide of claim 1.

29. The method of claim 29, wherein the PGC1 family of co-activator is selected from the group consisting of PRC, PGC1α and PGC1β.

30. A method for treating or preventing a disease or disorder in a subject in need thereof, the method comprising administering to the subject an effective amount of a composition comprising the peptide of claim 10.

31. A method for inhibiting the interaction between CBP80 and a PGC1 family of co-activator in a subject in need thereof, the method comprising administering to the subject an effective amount of a composition comprising the peptide of claim 10.
